(19) United States
(12) Patent Application Publication
(10) Pub. No.: US 2015/0267196 A1
(43) Pub. Date: Sep. 24, 2015

(54) BIODEGRADABLE HYDROGEL FOR POLYNUCLEOTIDE DELIVERY

(71) Applicant: CASE WESTERN RESERVE UNIVERSITY, Cleveland, OH (US)

(72) Inventors: Eben Alsberg, Cleveland, OH (US); Minh Khanh Nguyen, Cleveland, OH (US); Cong Huynh, Cleveland, OH (US); Alex Gilewski, Cleveland, OH (US); Matt Levy, Cleveland, OH (US); Vincent Rotello, Cleveland, OH (US); Gulen Yesilbag, Cleveland, OH (US); Oju Jeon, Cleveland, OH (US)

(21) Appl. No.: 14/435,378

(22) PCT Filed: Oct. 15, 2013
(86) PCT No.: PCT/US2013/065145

§ 371 Date: Apr. 13, 2015

Related U.S. Application Data
(60) Provisional application No. 61/713,276, filed on Oct. 12, 2012.

(51) Int. Cl. C12N 15/113

(52) U.S. Cl. CPC C12N 15/113; C12N 2310/351; C12N 2310/14

(57) ABSTRACT

A composition includes a biodegradable hydrogel that includes a hydrogel forming base polymer and a plurality of physiologically degradable ester linkages, amide linkages, azide-alkyne cycloaddition linkages, acrylate-thiol linkages, urethane linkages, and/or methacrylate-thiol linkages, a polynucleotide coupling polymeric molecule; and a polynucleotide. The polynucleotide is released under physiological conditions in a spatial and/or temporally controlled or predetermined manner from the composition.

### RELATED APPLICATION

This application claims priority from U.S. Provisional Application No. 61/713,276, filed Oct. 12, 2012, the subject matter of which is incorporated herein by reference in its entirety.

### TECHNICAL FIELD

The present invention generally relates to polynucleotide delivery, and disease therapeutics, and more particularly to biodegradable hydrogels.

### BACKGROUND

RNA interference is an efficient method to post-transcriptionally turn off the expression of specific proteins and transcription factors using small interfering RNA (siRNA) for cancer therapeutics or tissue engineering applications. Naked siRNA bears negative charges which limit its ability to passively diffuse across cell membranes and it is easily degraded by ribonucleases. To overcome these limitations, a number of siRNA delivery systems, including nanoparticles and microparticles, have been developed to deliver siRNA to treat a wide range of diseases. Unfortunately, these systems can be easily dispersed in vivo on account of their small size, making it difficult to locally target sites of interest for a prolonged period of time.

While, localized and sustained delivery is a promising strategy for siRNA delivery in vivo, there remains a need for injectable hydrogel systems that provide temporal control over the local delivery of siRNA to incorporated and surrounding cells.

### SUMMARY

Embodiments of the application described herein relate to compositions that can be used for polynucleotide delivery and tissue engineering, and more particularly to compositions that can provide localized, sustained, and/or controlled delivery of polynucleotides in a spatially and/or temporally controlled or predetermined manner at and/or on macroscale, mesoscale, microscale, or nanoscale level from the composition as well as related methods for using the composition in therapeutic applications.

In some embodiments the composition can include a biodegradable hydrogel, a polynucleotide coupling polymeric molecule, and/or a polynucleotide coupled to the polynucleotide coupling polymeric molecule. The biodegradable hydrogel can include a hydrogel forming base polymer and a plurality of physiologically degradable ester linkages, amide linkages, azide-alkyne cycloaddition linkages, acrylate-thiol linkages, urethane linkages, and/or methacrylate-thiol linkages. The hydrogel can be cytocompatible and, upon degradation, produce substantially non-toxic products. The polynucleotide coupling polymeric molecule can be coupled to the hydrogel forming base polymer. The polynucleotide can be released under physiological conditions in a spatial and/or temporally controlled or predetermined manner from the composition.

In some embodiments, the polynucleotide coupling polymeric molecule can be covalently linked to the hydrogel forming base polymer. For example, the polynucleotide coupling polymeric molecule being covalently linked to the hydrogel forming polymer by at least one of a covalent hydrolyzable ester, covalent hydrolyzable amide, covalent photodegradable urethane, covalent photodegradable and hydrolyzable ester, or covalent hydrolyzable acrylate-thiol linkage.

In other embodiments, the polynucleotide can be electrostatically coupled to the polynucleotide coupling polymeric molecule. The polynucleotide can also be covalently linked to the backbone of the polymeric macromers used to form the hydrogels.

In still other embodiments, the hydrogel and/or polynucleotide coupling polymeric molecule hydrogel can include ester bonds and urethane bonds directly linked to photolabile moieties. These bonds can be degradable by exposure to ultra-violet radiation. The hydrogel can also be degraded by hydrolysis of the ester linkages. Degradation of the bonds can be used to promote degradation of the hydrogel and/or spatial and/or temporal control of the release of the polynucleotide.

In other embodiments, the acrylated or methacrylated hydrogel forming base polymer can further include a plurality of physiologically degradable acrylate-thiol or methacrylate-thiol bonds.

In some embodiments, the hydrogel forming base polymer can be selected from the group consisting of dextran (DEX), polyethylene glycol (PEG) and poly(vinyl alcohol) (PVA). For example, the hydrogel forming base polymer can be selected from the group consisting of dextran-hydroxyethylmethacrylate (DEX-HEMA), dextran-mono-2-(acryloyloxy)ethyl succinate (DEX-MAES), polyvinyl alcohol-mono-2-(acryloyloxy)ethyl succinate (PVA-MAES), n-arm-polyethylene glycol-mono-2-(acryloyloxy)ethyl succinate (n-arm-PEG-MAES), n-arm-polyethylene glycol-acrylate (n-arm-PEG-A), n-arm-polyethylene glycol-thiol (n-arm-PEG-SH) and n-arm-polyethylene glycol-di(photolabile moiety-acrylate) [n-arm-PEG-D(PL-A)] glycol photolabile moiety-acrylate (n-arm-PEG-PL-A), n-arm-polyethylene glycol-azide (n-arm-PEG-azide), n-arm-polyethylene glycol-alkyne (n-arm-PEG-alkyne) (n=2, 4, 6, 8, 10, etc.).

The polynucleotide coupling polymeric molecule can be selected from the group consisting of poly(dimethylamino ethyl methacrylate) (pDMAEMA), poly(dimethylamino ethyl methacrylate-cysteamine) (poly(DMAEMA-co-cys)), linear or branched polyethyleneimine (PEI), polyethyleneimine-mono-2-(acryloyloxy)ethyl succinate (PEI-MAES), polyethyleneimine-thiol (PEI-thiol), and polyethyleneimine-glycidyl methacrylate (PEI-GMA), protamine, polylysine, polyamidoamine, polyethyleneimine-photolabile moiety-allyl (PEI-PL-allyl), polyethyleneimine-photolabile moiety-alkyne (PEI-PL-alkyne), polyethyleneimine-photolabile moiety-azide (PEI-PL-azide).

In a particular embodiment, the hydrogel forming polymer can include DEX-HEMA or DEX-MAES and the polynucleotide coupling molecule can include PEI-MAES or PEI-GMA or PEI-PL-allyl.

In some embodiments, the polynucleotide can be selected from the group consisting of DNA fragments, DNA plasmids, interfering RNA molecules. The polynucleotide can also include siRNA or miRNA. At least one cell can also be dispersed on or within or surrounding the biodegradable hydrogel. The at least one cell can be a progenitor cell.

In still other embodiments, the hydrogel can be photocrosslinked, formed in situ without or with chemicals or photo initiators, or formed via Click chemistry.

### BRIEF DESCRIPTION OF THE DRAWINGS

The foregoing and other features of the present invention will become apparent to those skilled in the art to which the present invention relates upon reading the following description with reference to the accompanying drawings, in which:

FIG. 1 illustrates (a) Synthesis of DEX-HEMA (left) and LPEI-GMA (right), and ¹H NMR spectra of (b) DEX-HEMA and c) LPEI-GMA in D₂O.

FIG. 2 is a schematic of hydrogel formation for delivery of siRNA to subsequently silence gene expression of encapsulated and surrounding HEK 293 cells.

FIG. 3 illustrates in vitro (a,b) swelling and (c,d) degradation of (a,c) 8% w/w and (b,d) 12% w/w DEX hydrogels with different LPEI concentrations.

FIG. 4 illustrates storage (G′) and loss (G″) moduli of 12% w/w DEX hydrogels with different LPEI concentrations with or without siRNA (26.6 μg/100 μl gel solution) (N=3). Filled and unfilled symbols represent G′ and G″, respectively. Circle, DEX+10PEI with siRNA; triangle down, DEX-only with siRNA; diamond, DEX+10PEI; square, DEX+5PEI; triangle up, DEX-only.

FIG. 5 illustrates the viability of (a) cells surrounding and (b) encapsulated within 12% w/w DEX hydrogels with various LPEI concentrations as measured using a 3-(4,5-dimethylthiazol-2-yl)-5-(3-carboxymethoxyphenyl)-2-(4-sulfophenyl)-2H-tetrazolium, inner salt (MTS) assay.

FIG. 6 illustrates siRNA release profiles from (a) 8% w/w DEX hydrogels with siRNA loading of 13.3 μg (*p<0.05 compared with DEX+5PEI and DEX+10PEI, #p<0.05 compared with DEX+10PEI), (b) 12% w/w DEX hydrogels with siRNA loading of 13.3 μg (*p<0.05 compared with DEX+5PEI and DEX+10PEI, #p<0.05 compared with DEX+5PEI), and (c) 12% w/w DEX hydrogels with siRNA loading of 26.6 μg (*p<0.05 compared with DEX+10PEI). Insets indicate siRNA release rate (μg/day) from the corresponding hydrogels.

FIG. 7 illustrate the percentage of positive deGFP HEK293 cells after exposure to released media from 12% w/w DEX-LPEI hydrogels originally containing (a) 13.3 μg (*p<0.05 compared with day 3 of DEX-only, **p<0.05 compared with day 3 of DEX+5PEI, ***p<0.05 compared with day 14 of DEX-only, #p<0.05 compared with day 7 of DEX+5PEI, ##p<0.05 compared with day 14 of DEX+5PEI, and ###p<0.05 compared with day 14 of DEX+10PEI), or (b) 26.6 μg siRNA (*p<0.05 compared with day 3 of DEX-only, **p<0.05 compared with day 7 of DEX-only, ***p<0.05 compared with day 14 of DEX-only). Samples are normalized to release samples from hydrogels without any siRNA. GFP expression of cells exposed to all release samples (except those exposed to release samples from DEX-only by day 14 in FIG. 7a) was significantly different compared with the controls (No siRNA) at corresponding time points.

FIG. 8 illustrates the confocal fluorescent photomicrographs of deGFP HEK293 cells encapsulated in 3D hydrogels with or without siRNA (Group 1: DEX-only without siRNA, Group 2: DEX-only with siRNA, Group 3: DEX+10PEI with siRNA). Control hydrogels without siRNA treatment contained cells with strong deGFP expression at all time points. deGFP expression of cells in the DEX-only hydrogels with siRNA was decreased at day 3, but strong expression returned at day 7 and day 11. Substantial knockdown of deGFP expression was observed at all time points for cells in the DEX+10PEI hydrogels. The scale bar indicates 200 μm.

FIG. 9 illustrates a) swelling, b) degradation, c) rheology properties of hydrogels, d) release of siRNA/PEI complexes from hydrogels, e) schematic figure of incorporation of siRNA/PEI complexes into hydrogels, f) bioactivity of siRNA/PEI released from M, MA and A gels compared to that of fresh siRNA/PEI complexes and controls.

FIG. 10 illustrates a) schematic figure demonstrating RNA and hMSCs encapsulation into hydrogels and hMSCs differentiation, b) noggin gene expression, c) ALP activity, d) Runx2, e) BSP and f) PPAR-γ gene expression in hMSCs encapsulated within hydrogels. *p<0.05 compared with Control, **p<0.05 compared with siNoggin and Cotransfection, and ***p<0.05 compared with Cotransfection at specific time point.

FIG. 11 illustrates a) calcium content in hydrogels, b) mineralization in hydrogels stained with Alizarin red. *p<0.05 compared with Control, **p<0.05 compared with siNoggin and Cotransfection, and ***p<0.05 compared with Cotransfection at specific time point.

FIG. 12 illustrates a schematic drawing of a reaction scheme for forming an in situ formed biodegradable hydrogel in accordance with one embodiment.

FIG. 13 illustrates a schematic drawing of a reaction scheme for forming an in situ formed biodegradable hydrogel in accordance with another embodiment.

### DETAILED DESCRIPTION

Methods involving conventional molecular biology techniques are described herein. Such techniques are generally known in the art and are described in detail in methodology treatises, such as Current Protocols in Molecular Biology, ed. Ausubel et al., Greene Publishing and Wiley-Interscience, New York, 1992 (with periodic updates). Unless otherwise defined, all technical terms used herein have the same meaning as commonly understood by one of ordinary skill in the art to which the present invention pertains. Commonly understood definitions of molecular biology terms can be found in, for example, Rieger et al., Glossary of Genetics: Classical and Molecular, 5th Ed., Springer-Verlag: New York, 1991, and Lewin, Genes V, Oxford University Press: New York, 1994. The definitions provided herein are to facilitate understanding of certain terms used frequently herein and are not meant to limit the scope of the present invention.

As used herein, the terms “biodegradable” and “bioresorbable” may be used interchangeably and refer to the ability of a material (e.g., a natural polymer or macromer) to be fully resorbed in vivo. “Full” can mean that no significant extracellular fragments remain. The resorption process can involve elimination of the original implant material(s) through the action of body fluids, enzymes, cells, and the like.

As used herein, the term “polynucleotide” can refer to oligonucleotides, nucleotides, or to a fragment of any of these, to DNA or RNA (e.g., mRNA, rRNA, tRNA, miRNA, siRNA) of genomic or synthetic origin which may be single-stranded or double-stranded and may represent a sense or antisense strand, to peptide nucleic acids, or to any DNA-like or RNA-like material, natural or synthetic in origin, including, e.g., iRNA, ribonucleoproteins (e.g., iRNPs). The term can also encompass nucleic acids (i.e., oligonucleotides) containing known analogues of natural nucleotides, as well as nucleic acid-like structures with synthetic backbones.

As used herein, the term “cell” can refer to any progenitor cell, such as totipotent stem cells, pluripotent stem cells, and multipotent stem cells, as well as any of their lineage descendant cells, including more differentiated cells. The terms “stem cell” and “progenitor cell” are used interchangeably herein. The cells can derive from embryonic, fetal, or adult tissues. Examples of progenitor cells can include totipotent stem cells, multipotent stem cells, mesenchymal stem cells (MSCs), hematopoietic stem cells, neuronal stem cells, hematopoietic stem cells, pancreatic stem cells, cardiac stem cells, embryonic stem cells, embryonic germ cells, neural crest stem cells, kidney stem cells, hepatic stem cells, lung stem cells, hemangioblast cells, and endothelial progenitor cells. Additional exemplary progenitor cells can include de-differentiated chondrogenic cells, chondrogenic cells, cord blood stem cells, multi-potent adult progenitor cells, myogenic cells, osteogenic cells, tendogenic cells, ligamentogenic cells, adipogenic cells, and dermatogenic cells.

As used herein, the term “subject” can refer to any animal, including, but not limited to, humans and non-human animals (e.g., rodents, arthropods, insects, fish (e.g., zebrafish)), non-human primates, ovines, bovines, ruminants, lagomorphs, porcines, caprines, equines, canines, felines, ayes, etc.), which is to be the recipient of a particular treatment. Typically, the terms “patient” and “subject” are used interchangeably herein in reference to a human subject.

As used herein, “tissue” is typically an aggregate of cells of the same origin, but may be an aggregate of cells of different origins. The cells can have the substantially same or substantially different function, and may be of the same or different type. “Tissue” can include, but is not limited to, an organ, a part of an organ, bone, cartilage, skin, neuron, axon, blood vessel, cornea, muscle, fascia, brain, prostate, breast, endometrium, lung, pancreas, small intestine, blood, liver, testes, ovaries, cervix, colon, stomach, esophagus, spleen, lymph node, bone marrow, kidney, peripheral blood, embryonic, or ascite tissue.

As used herein, the terms “inhibit,” “silencing,” and “attenuating” can refer to a measurable reduction in expression of a target mRNA (or the corresponding polypeptide or protein) as compared with the expression of the target mRNA (or the corresponding polypeptide or protein) in the absence of an interfering RNA molecule of the present invention. The reduction in expression of the target mRNA (or the corresponding polypeptide or protein) is commonly referred to as “knock-down” and is reported relative to levels present following administration or expression of a non-targeting control RNA.

This application generally relates to polynucleotide delivery used in tissue engineering and therapeutic applications, and more particularly to compositions that can provide localized, sustained, and/or controlled delivery of polynucleotides in a spatially and/or temporally controlled or predetermined manner at and/or on macroscale, mesoscale, microscale, or nanoscale level from the composition. The composition can include a biodegradable hydrogel, a polynucleotide coupling polymeric molecule, and/or a polynucleotide coupled to the polynucleotide coupling polymeric molecule as well as to methods for using the compositions in different polynucleotide delivery, tissue engineering and/or therapeutic applications. The biodegradable hydrogel described herein is substantially cytocompatible (i.e., substantially non-cytotoxic) and can includes controllable physical properties, such as degradation rate, swelling behavior, and tunable polynucleotide release profiles, which allow the polynucleotide to be released under physiological conditions in a spatially and/or temporally controlled or predetermined manner from the composition.

In some embodiments, the biodegradable hydrogel can include an acrylated or methacrylated hydrogel forming base polymer and a plurality of physiologically degradable ester linkages, amide linkages, azide-alkyne cycloaddition linkages, acrylate-thiol linkages, urethane linkages, and/or methacrylate-thiol linkages. The hydrogel can be cytocompatible and, upon degradation, producing substantially non-toxic products and/or allow spatial and/or temporal control of the release of the polynucleotide.

The methacrylated and/or acrylated base polymer can include at least one methacrylate group, or acrylate group that can be linked, polymerized, and/or cross-linked to another polymer, macromer, or oligomer, and/or the polynucleotide coupling polymeric molecule and/or base polymer.

In some embodiments, the hydrogel forming base polymer can include at least one of dextran (DEX), polyethylene glycol (PEG) and/or poly(vinyl alcohol) (PVA). The methacrylated and/or acrylated base polymer can be, for example, selected from the group consisting of dextran-hydroxyethylmethacrylate (DEX-HEMA), dextran-mono-2-(acryloyloxy)ethyl succinate (DEX-MAES), polyvinyl alcohol-mono-2-(acryloyloxy)ethyl succinate (PVA-MAES), n-arm-polyethylene glycol-mono-2-(acryloyloxy)ethyl succinate (n-arm-PEG-MAES), n-arm-polyethylene glycol-acrylate (n-arm-PEG-A), n-arm-polyethylene glycol-thiol (n-arm-PEG-SH) and n-arm-polyethylene glycol-di(photolabile moiety-acrylate) [n-arm-PEG-D(PL-A)] glycol photolabile moiety-acrylate) (n-arm-PEG-PL-A), n-arm-polyethylene glycol-azide (n-arm-PEG-azide), n-arm-polyethylene glycol-alkyne (n-arm-PEG-alkyne) (n=2, 3, 4, 5, 6, 7, 8, 10, etc.)

In some embodiments, the hydrogel forming base polymer can be formed by reacting an acrylate or methacrylate group with a hydrogel forming base polymer or oligomer to form a plurality of methacrylate and/or acrylate substituted macromers. The methacrylated or acrylated hydrogel forming base polymer can also include a plurality of physiologically degradable acrylate-thiol or methacrylate-thiol bonds

An example of an acrylated hydrogel forming base polymer has the following Formula I:

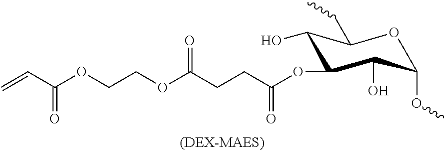

DEX-MAES can be synthesized from mono(2-acryloyloxy)ethyl succinate (MAES) and dextran (DEX) as shown below:

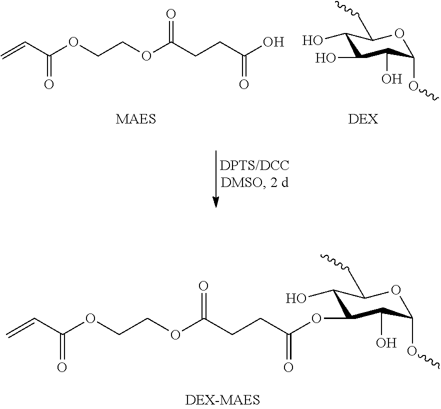

Another example of an acrylated hydrogel forming base polymer can have the following Formula II:

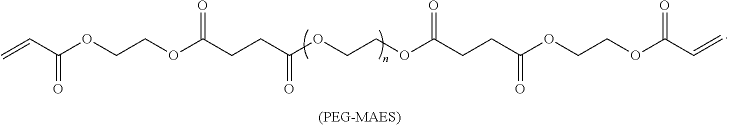

PEG-MAES can be synthesized from poly(ethylene glycol) and mono(2-acryloyloxy)ethyl succinate (MAES).

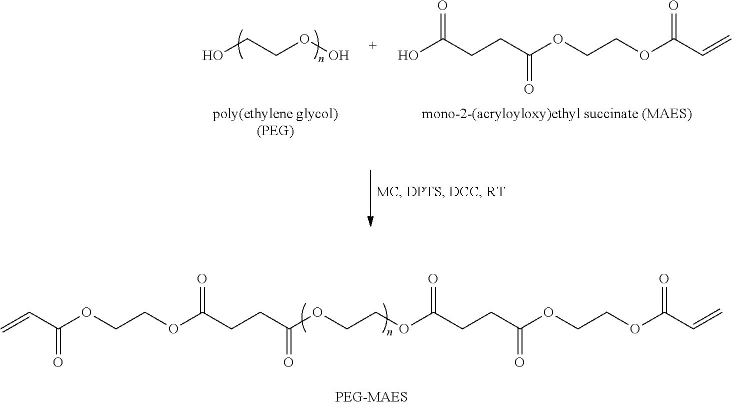

A further example of an acrylated hydrogel forming base polymer can include an oligomer, which has the following Formula III:

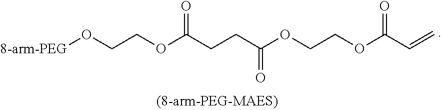

8 arm-PEG-MAES can be synthesized from 8-arm-poly(ethylene glycol) and mono(2-acryloyloxy)ethyl succinate (MAES).

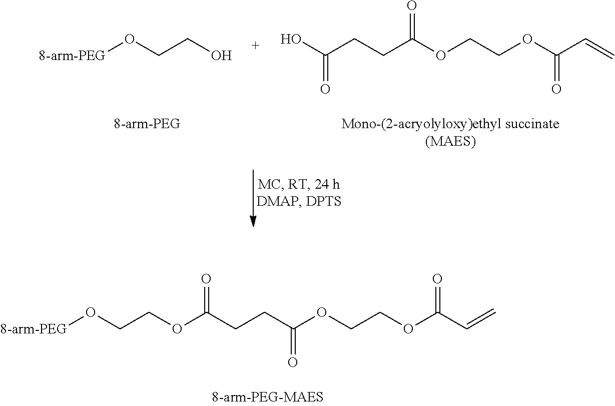

A still further example of an acrylated hydrogel forming base polymer can include an oligomer which can also have the following Formula IV:

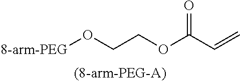

8 arm-PEG-A can be synthesized from 8-arm-poly(ethylene glycol) and acryloyl chloride (AC) as shown below.

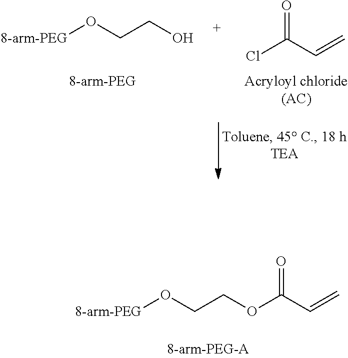

An acrylated hydrogel forming base polymer can also have the following Formula V:

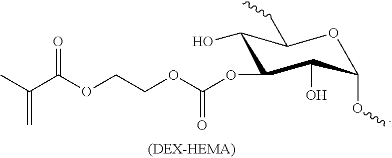

DEX-HEMA can be synthesized from hydroxyethylmethacrylate-IC (HEMA-IC) and dextran (DEX).

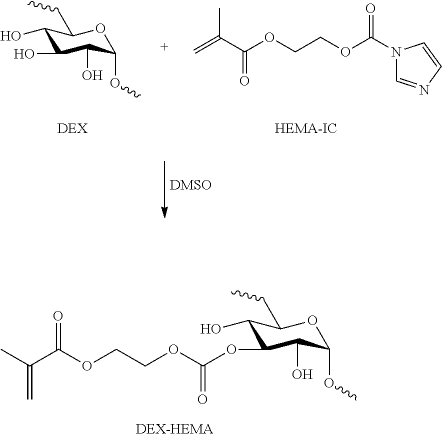

Another example of an acrylated hydrogel forming base polymer can have the following Formula VI:

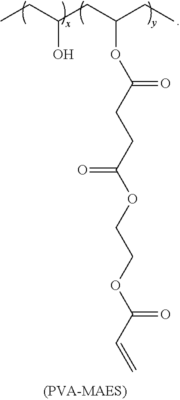

PVA-MAES for use in the invention can be synthesized from poly(vinyl alcohol)(PVA) and mono(2-acryloyloxy)ethyl succinate (MAES).

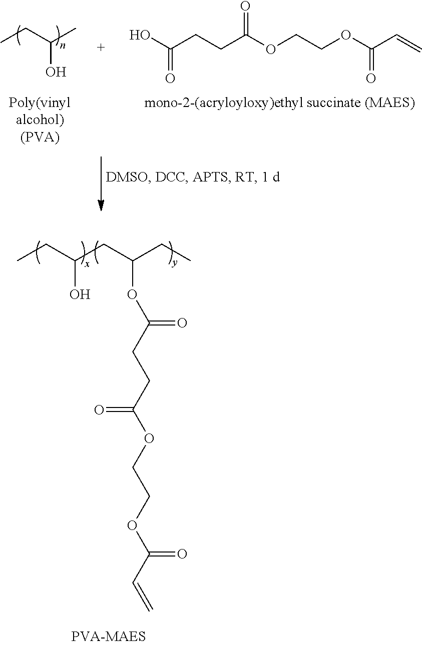

The composition can further include a polynucleotide coupling polymeric molecule that can be part of, covalently linked to, or coupled to the hydrogel base polymer. The polynucleotide coupling polymer molecule can include any polymeric molecule that can complex (electrostatically couple) with and/or be ionically linked to the polynucleotide.

In certain aspects, the polynucleotide coupling polymeric molecule can be covalently linked to the biodegradable hydrogel forming methacrylated or acrylated base polymer. For example, the polynucleotide coupling polymeric molecule can be covalently linked to the hydrogel forming polymer by at least one of a covalent hydrolyzable ester, covalent hydrolyzable amide, covalent photodegradable urethane, covalent photodegradable and hydrolyzable ester, or covalent hydrolyzable acrylate-thiol linkage. In other embodiments, the polynucleotide coupling polymeric molecule can be provided in and/or on and/or mixed with the hydrogel so the hydrogel contains or encapsulates the polynucleotide coupling polymeric molecule to, for example, control spatial and/or temporal release of the polynucleotide coupling polymeric molecule and/or polynucleotide upon degradation the hydrogel.

Polynucleotide coupling polymeric molecules can be made with a variety of polymers and are capable of binding to or electrostatically coupling to polynucleotides such as DNA and/or siRNA and/or miRNA. It will be appreciated that the cationic polymeric molecule may also have similar or identical material compositions as the biodegradable hydrogel forming methacrylated or acrylated base polymer.

An example of a polynucleotide coupling polymeric molecule that can be covalently linked to a hydrogel forming base polymer has the following Formula VII:

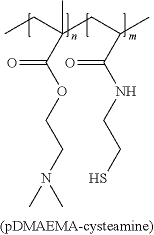

pDMAEMA-cysteamine can be synthesized from p(DMAEMA-co-NASI) and cysteamine as shown below.

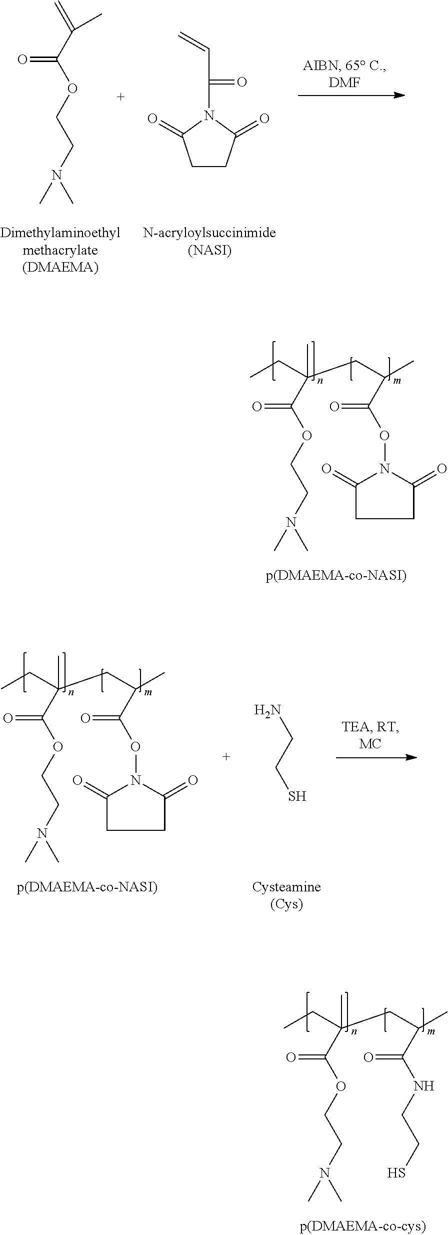

In some aspects, the biodegradable hydrogel includes acrylated dextran or PEG covalently linked to pDMAEMA-cysteamine.

In another example, the cationic polynucleotide coupling polymeric molecule that can be covalently linked to a hydrogel forming methacrylated or acrylated base polymer has the following Formula VIII:

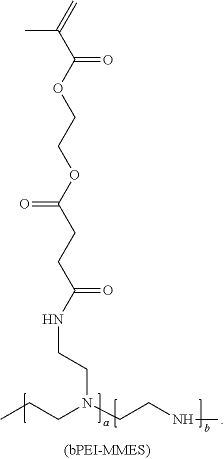

bPEI-MMES can be synthesized from branched polyethyleneimine (bPEI) and mono(2-methacryloyloxy)ethyl succinate (MMES) as shown below.

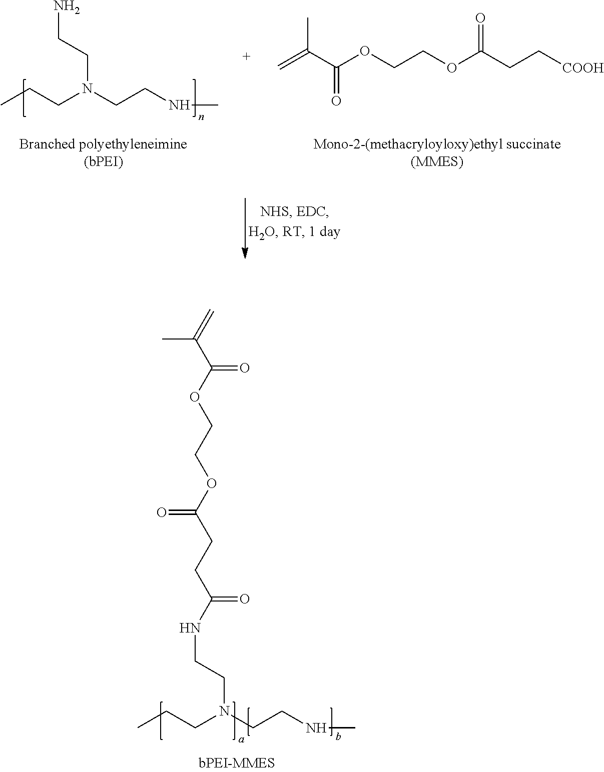

In an exemplary embodiment, a photocrosslinked biodegradable hydrogel includes DEX-HEMA covalently linked to bPEI-MMES through a hydrolysable ester linkage.

In another example, the cationic polynucleotide coupling polymeric molecule that can be covalently linked to a hydrogel forming methacrylated or acrylated base polymer has the following Formula IX:

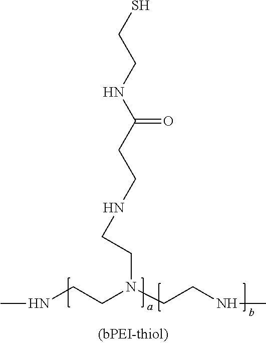

bPEI-thiol can be synthesized from branched polyethyleneimine (bPEI) and N,N′-cystaminebisacrylamide (CBA) as shown below.

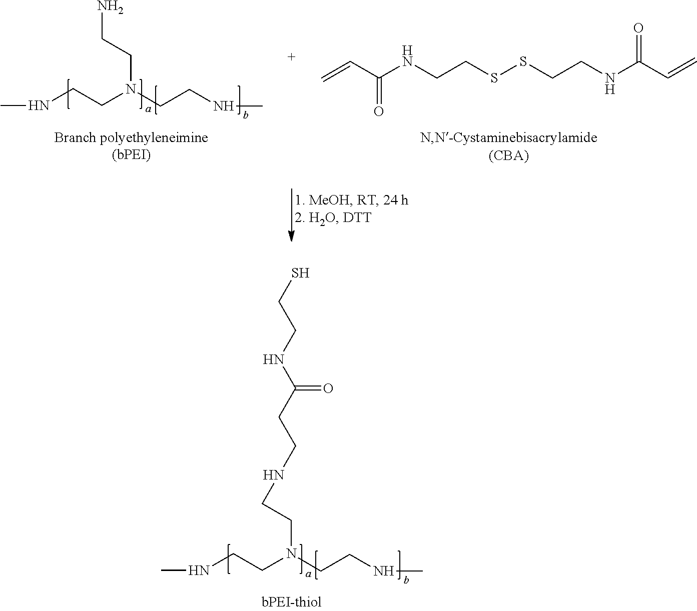

In some aspects, a biodegradable hydrogel includes acrylated PEG covalently linked to bPEI-thiol.

In some embodiments, a biodegradable hydrogel that is coupled to a polynucleotide coupling polymeric molecule or that includes the polynucleotide coupling polymeric molecule can be formed by photocrosslinking, formed in situ without or with chemicals or photoinitiators, or formed via click chemistry (e.g., by copper-assisted or copper free azide-alkyne cycloaddition). In one example, biodegradable hydrogels that are linked to the polynucleotide coupling polymeric molecule can be formed by photocrosslinking a methacrylated or acrylated hydrogel forming base polymer with a polymeric molecule/polynucleotide complex using UV light in the presence of photoinitiators. For example, a methacrylated or acrylated hydrogel forming base polymer can be photocrosslinked with a cationic polynucleotide coupling polymeric molecule by first dissolving a desired amount of the methacrylated or acrylated hydrogel forming polymer in an appropriate amount of diH₂O or aqueous media (e.g., PBS) containing a desired amount of a photoinitiator (e.g., Irgacure D2959), and then a polynucleotide coupling polymeric molecule and polynucleotide can then be added to the solution. In some aspects the polynucleotide coupling polymeric molecule and polynucleotide can be combined in solution prior to be added to the acrylated hydrogel forming polymer solution and/or photoinitiator.

The solution can then be injected into a curing vessel (e.g., a 96 well plate) and exposed to a light source at a wavelength and for a time to promote cross-linking of the acrylate groups of the polymers and form the photocrosslinked biodegradable hydrogel. For example, the polymers can be exposed to UV light of about 320-500 nm at about 3.5 mW cm⁻² for about 85 seconds using an Omnicure S 1000 UV Spot Cure System (Lumen Dynamics Group, Mississauga, Ontario, Canada) to form the hydrogel.

A photoinitiator can include any photo-initiator that can initiate or induce polymerization of the liquid acrylic or acrylate monomer. Examples of the photoinitiator can include camphorquinone, benzoin methyl ether, 2-hydroxy-2-methyl-1-phenyl-1-propanone, diphenyl(2,4,6-trimethylbenzoyl)phosphine oxide, benzoin ethyl ether, benzophenone, 9,10-anthraquinone, ethyl-4-N,N-dimethylaminobenzoate, diphenyliodonium chloride and derivatives thereof.

In other examples, a biodegradable hydrogel can be formed by in situ forming using a Michael reaction between an acrylated/methacrylated macromer with a thiolated macromer. The acrylated/methacrylated macromers can be n-arm poly(ethylene glycol) (PEG)-acrylate/methacrylate, n-arm-PEG-photolabile moiety-acrylate (n=2, 3, 4, 5, 6, 7, 8, 10, etc.), acrylated/methacrylated poly(vinyl alcohol) (PVA) and acrylated/methacrylated natural polymers (such as dextran, chitosan, alginate, gelatin). The thiolated macromers can be n-arm PEG-thiol (n=2, 3, 4, 5, 6, 7, 8, etc.), thiolated natural polymers (such as dextran, chitosan, alginate, gelatin). The polynucleotide coupling polymeric molecule can be protamine, polylysine, polyamidoamine, branched polyethyleneimine (PEI), branched polyethyleneimine-cysteamine (PEI-co-Cys), or linear polyethyleneimine As illustrated in Example 2 below, in situ forming hydrogels can prepared by mixing a acrylated/methacrylated macromer solution with a thiolated macromer solution in aqueous solution at pH 7.0-8.0 and the combining or reacting the hydrogel with polynucleotide coupling polymeric molecule that is coupled, complexed, or linked to the polynucleotide.

The polynucleotide that is coupled to, complexed with, and/or covalently linked to the polynucleotide coupling polymeric molecule and/or hydrogel can be any polynucleotide capable of modulating a function and/or characteristic of a cell. For example, the polynucleotide may be capable of modulating a function and/or characteristic of a cell that is dispersed on or within the biodegradable hydrogel. Alternatively or additionally, the polynucleotide may be capable of modulating a function and/or characteristic of an endogenous cell surrounding the composition implanted in a tissue defect, for example, and guide the cell into the defect.

In some embodiments, the polynucleotide coupling polymeric molecule can be covalently conjugated with a hydrogel forming methacrylated or acrylated polymer described such that the swelling or degradation properties of the hydrogels are not affected, and the addition of siRNA and a polynucleotide coupling polymeric molecule has minimal effect on the mechanical properties of a hydrogel of the invention. It is contemplated that a polynucleotide, (e.g., siRNA) released from the hydrogel exhibits high bioactivity with cells surrounding and inside the hydrogels over an extended period of time. The controllable and sustained delivery of a polynucleotide using a hydrogel allows for tailored release profiles for use in guiding cell fate in regenerative medicine and other therapeutic applications such as cancer treatment.

For example, localized delivery can allow for targeted siRNA or miRNA (etc.) exposure to non-malignant tumors or sites of tumor resection, which may lower the dose required for efficacy and potentially reduce effects on non-target cells. The delivery of siRNA at a specific location in the body may also permit regulation of transplanted or host cell gene expression to aid in the regeneration of damaged or diseased tissues. In addition, sustained delivery of siRNA may provide a silencing effect over an extended period of time.

The at least one polynucleotide encoding or comprising, for example, transcription factors, differentiation factors, growth factors, and combinations thereof. Examples of polynucleotides include DNA fragments, DNA plasmids, interfering RNA molecules, such as siRNAs, DNA encoding for an shRNA of interest, and oligonucleotides. In other embodiments, the hydrogel can include other bioactive agents.

In some embodiments, the polynucleotide can be electrostatically linked to the polynucleotide coupling polymeric molecule of the biodegradable hydrogel. For example, a cationic polynucleotide coupling polymeric molecule can electrostatically interact with negatively charged siRNA to maintain siRNA within the hydrogel. Degradation of the covalent linkages between the cationic polynucleotide coupling polymeric molecule and the hydrogel forming polymer leads to tunable spatial and/or temporal release of the polynucleotide coupling polymeric molecule/polynucleotide complexes over time.

In other embodiments, the polynucleotide can be covalently linked to the hydrogel forming polymer by at least one of a covalent hydrolyzable ester, covalent hydrolyzable amide, covalent photodegradable urethane, covalent photodegradable and hydrolyzable ester, or covalent hydrolyzable acrylate-thiol linkage. For example, as shown below, thiolated siRNA can be coupled to DEX-thiol by a disulfide bond to form a hydolyzable linkage.

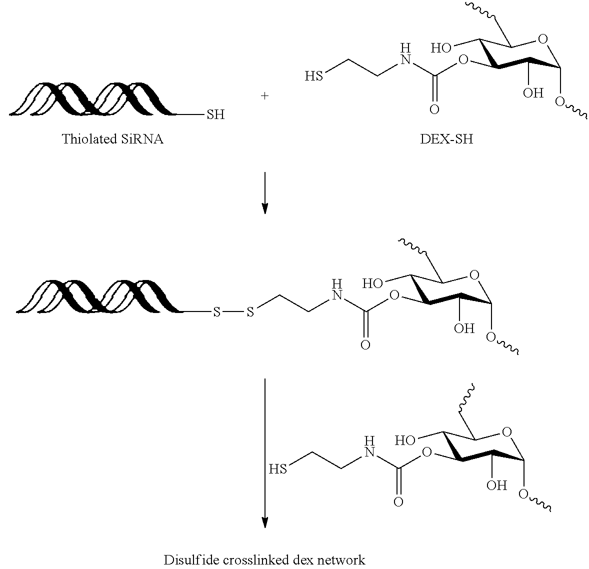

In still other embodiments, as shown below, thiolated siRNA can be coupled to thiolated DEX by a disulfide bond to form a thiolated DEX-siRNA compound that can then cross-link with thiolated dextran to form a hydrogel.

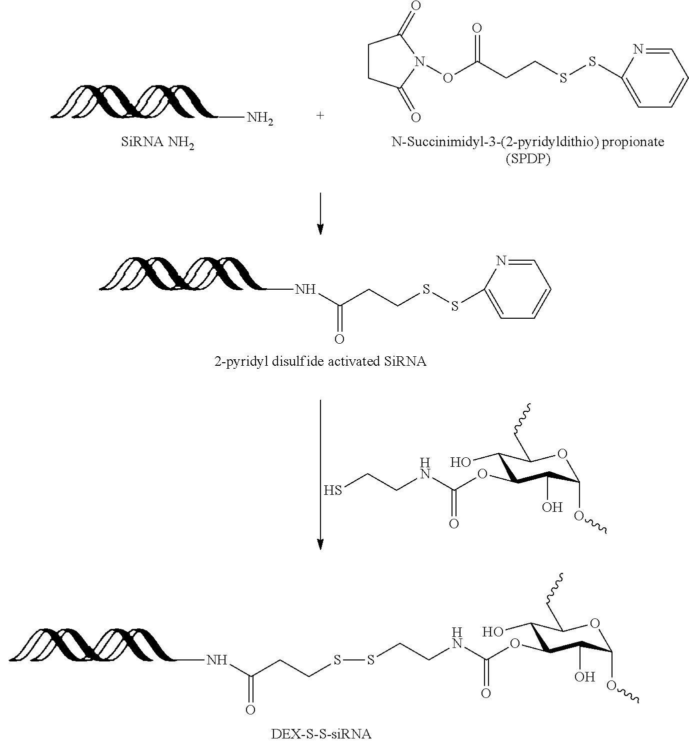

In other embodiments, as shown below, the polynucleotide can react with N-succinmidyl-3-(2-pyridylthio)propionate to form 2-pyridyl disulfide polynucleotide that can then react with DEX-thiol to form a hydrogel.

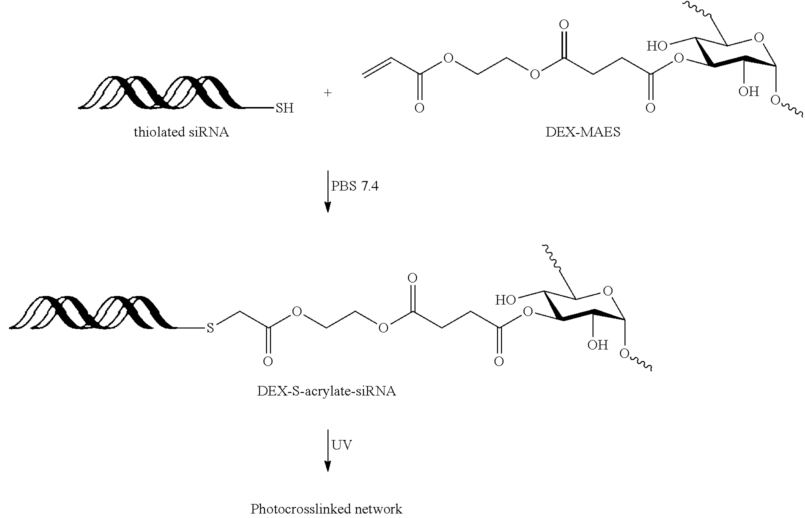

In still other embodiments, as shown below, thiolated siRNA can be coupled to DEX-MAES by a thiol-acrylate bond to form a DEX-S-acrylate siRNA that can then crosslink by UV to form a hydrogel.

In certain embodiments, a polynucleotide can comprise an interfering RNA molecule incorporated on or within at least one carrier material dispersed on or within the photocrosslinked biodegradable hydrogel. The interfering RNA molecule can include any RNA molecule that is capable of silencing a target mRNA and thereby reducing or inhibiting expression of a polypeptide encoded by the target mRNA. Alternatively, the interfering RNA molecule can include a DNA molecule encoding for a shRNA of interest. For example, the interfering RNA molecule can comprise a short interfering RNA (siRNA) or microRNA molecule capable of silencing a target mRNA that encodes any one or combination of the polypeptides or proteins described above.

In another aspect, the biodegradable hydrogel can include first and second (and/or third, fourth, etc.) polynucleotides loaded on or within the hydrogel. The first and second polynucleotides may comprise the same or different polynucleotides. The first and second polynucleotides can be differentially, sequentially, and/or controllably released from the biodegradable hydrogel to modulate a different function and/or characteristic of a cell. It will be appreciated that the first polynucleotide can have a release profile that is the same or different from the release profile of the second polynucleotide from the biodegradable hydrogel. The first and second polynucleotide may be dispersed uniformly on or within the photocrosslinked biodegradable hydrogel or, alternatively, dispersed such that different densities of the polynucleotides are localized on or within different portions of the hydrogel.

The amount or percentage of hydrogel forming base polymer, polynucleotide and polynucleotide coupling polymeric molecules in a biodegradable hydrogel of the invention can varied in order to control the mechanical properties, swelling ratios, degradation profiles of the hydrogels and/or polynucleotide release profiles.

It is contemplated that the degradation of the hydrogel and/or covalent linkages (e.g., hydrolysable ester linkages) between the cationic polymeric molecules/polynucleotide complex and the base polymer in vivo allows the hydrogel to readily biodegrade and be used for in vivo applications described below.

The biodegradable hydrogel can also include at least one photolabile bond that can degrade by exposure to UV light. The photolabile bonds can be provided uniformly throughout the hydrogel or in selected regions or portions of the hydrogel and/or polynucleotide coupling polymeric molecule to allow spatial and/or temporal control the release of the polynucleotide upon exposure to UV light. The photolabile moieties can be directly linked to ester bonds and/or urethane bonds of the hydrogel and/or polynucleotide coupling polymeric molecule. For example, the photolabile moiety can include a thiol acrylate bond that is photolabile upon exposure to UV light.

In some embodiments, the hydrogel and/or polynucleotide coupling polymeric molecule can be dually cross-linked with a first hydrolysable and photolabile moiety and a second disulfide bond moiety to control the release of polynucleotide with minimum dependence on the gel degradation rate. In other embodiments, the gels can be degraded by cells naturally or with peptides sequences that can be degraded by cell secreted enzymes.

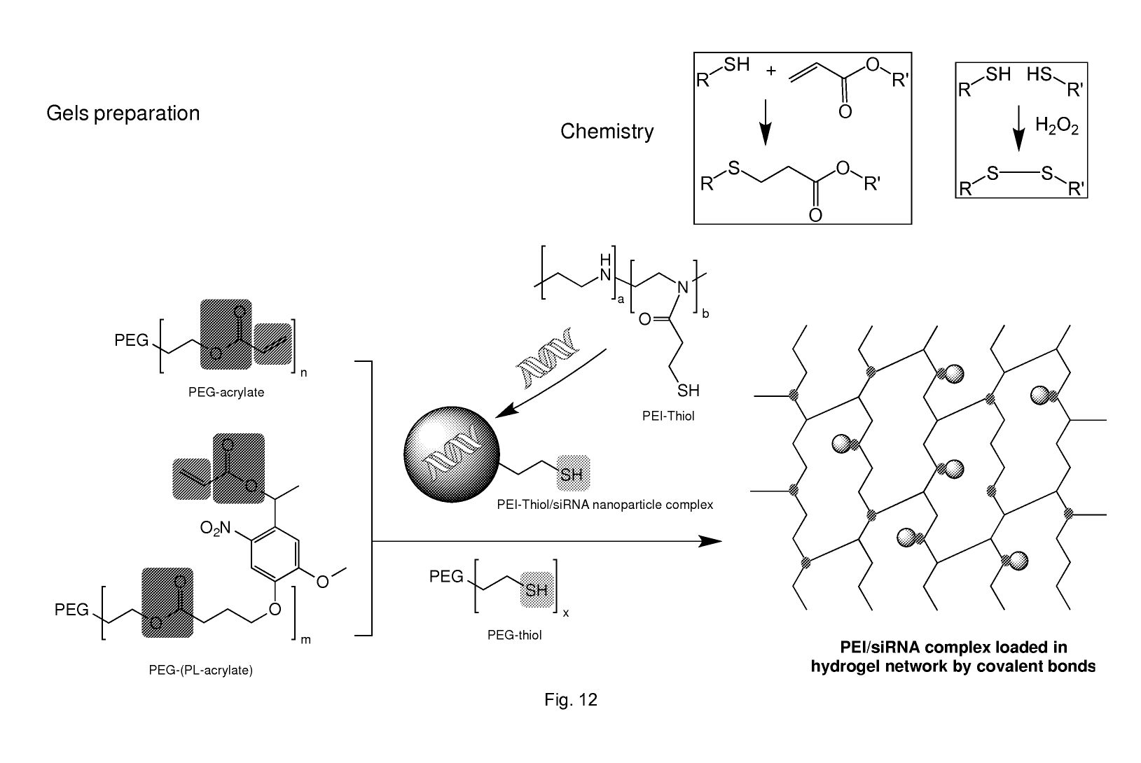

For example, as shown in FIG. 12, an in situ photolabile hydrogel can be prepared by the by a Michael reaction between acrylate groups in multi-arm-PEG-acrylate and multi-arm-PEG-photolabile-acrylate and thiol groups in multi-arm-PEG-thiols with or without H₂O₂ (H₂O₂ is the catalyst for forming disulfide bonds). The hydrogels can be degraded via ester bonds in multi-arm-PEG-acrylate and photolabile moieties (PL) in multi-arm-PEG-photolabile-acrylate and disulfide bonds. The degradation rate can be controlled by exposing UV and/or changing the density of disulfide bond which has low degradation rate. PEI, which can form nanocomplex with siRNA, can also modified with thiol groups that can react with acrylate groups in multi-arm-PEG-photolabile-acrylate prior gelation to form covalent bonds with hydrogel network for controlled siRNA release by UV degradation. The advantage of this hydrogel system is partly controlled siRNA release by UV degradation to release PEI-siRNA nanocomplex.

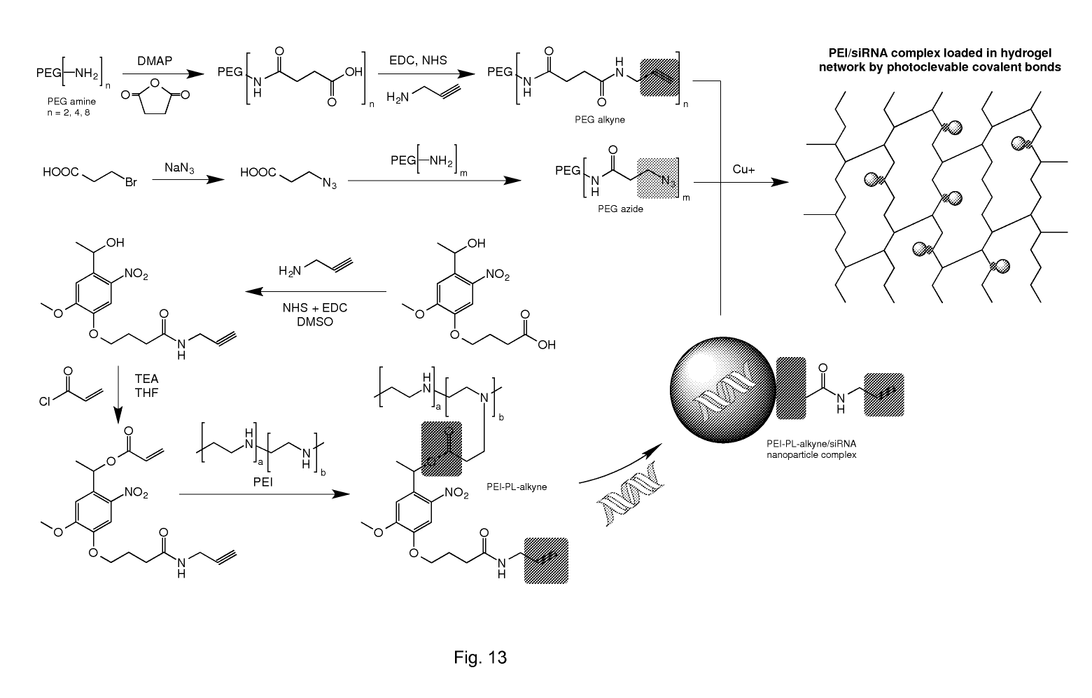

In another example, as shown in FIG. 13, an in situ photolabile hydrogel can be prepared by a Click reaction between alkyne groups in multi-arm-PEG-alkyne and azide groups in multi-arm-PEG-azide in the present of Cu⁺ as a catalyst. The multi-arm-PEG macromer backbone can be also replaced by other macromers such as polysaccarides. PEI, which can form nanocomplex with siRNA, was modified with photolabile moiety (PL) and alkyne (or azide) groups that can make covalent bonds with hydrogel network for controlled siRNA release by UV degradation The advantage of this hydrogel system is fully controlled siRNA release by UV degradation to release PEI-siRNA nanocomplex.

In another aspect, the biodegradable hydrogel can include at least one cell dispersed on or within the hydrogel. For example, cells can be entirely or partly encapsulated within the biodegradable hydrogel. Cells can include any progenitor cell, such as a totipotent stem cell, a pluripotent stem cell, or a multipotent stem cell, as well as any of their lineage descendant cells, including more differentiated cells (described above), such as MSCs.

The cells can be autologous, xenogeneic, allogeneic, and/or syngeneic. Where the cells are not autologous, it may be desirable to administer immunosuppressive agents in order to minimize immunorejection. The cells employed may be primary cells, expanded cells, or cell lines, and may be dividing or non-dividing cells. Cells may be expanded ex vivo prior to introduction into or onto the biodegradable hydrogel. For example, autologous cells can be expanded in this manner if a sufficient number of viable cells cannot be harvested from the host subject. Alternatively or additionally, the cells may be pieces of tissue, including tissue that has some internal structure. The cells may be primary tissue explants and preparations thereof, cell lines (including transformed cells), or host cells.

Generally, cells can be introduced into the biodegradable hydrogel in vitro, although in vivo seeding approaches can optionally or additionally be employed. Cells may be mixed with the biodegradable hydrogel and cultured in an adequate growth (or storage) medium to ensure cell viability. Alternatively, the cells can be added to the solution of hydrogel forming macromers and polynucleotide and then the mixtures can be crosslinked. If the biodegradable hydrogel is to be implanted for use in vivo after in vitro seeding, for example, sufficient growth medium may be supplied to ensure cell viability during in vitro culture prior to in vivo application. Once the biodegradable hydrogel has been implanted, the nutritional requirements of the cells can be met by the circulating fluids of the host subject.

Any available method may be employed to introduce the cells into the biodegradable hydrogel. For example, cells may be injected into the biodegradable hydrogel (e. g., in combination with growth medium) or may be introduced by other means, such as pressure, vacuum, osmosis, or manual mixing. Alternatively or additionally, cells may be layered on the biodegradable hydrogel, or the hydrogel may be dipped into a cell suspension and allowed to remain there under conditions and for a time sufficient for the cells to incorporate within or attach to the hydrogel. Generally, it is desirable to avoid excessive manual manipulation of the cells in order to minimize cell death during the impregnation procedure. For example, in some situations it may not be desirable to manually mix or knead the cells with the biodegradable hydrogel; however, such an approach may be useful in those cases in which a sufficient number of cells will survive the procedure. Cells can also be introduced into the biodegradable hydrogel in vivo simply by placing the hydrogel in the subject adjacent a source of desired cells. Bioactive agents released from the biodegradable hydrogel may also recruit local cells, cells in the circulation, or cells at a distance from the implantation or injection site.

As those of ordinary skill in the art will appreciate, the number of cells to be introduced into the biodegradable hydrogel will vary based on the intended application of the hydrogel and on the type of cell used. Where dividing autologous cells are being introduced by injection or mixing into the biodegradable hydrogel, for example, a lower number of cells can be used. Alternatively, where non-dividing cells are being introduced by injection or mixing into the biodegradable hydrogel, a larger number of cells may be required. It should also be appreciated that the macromer scaffold can be in either a hydrated or lyophilized state prior to the addition of cells. For example, the macromer scaffold can be in a lyophilized state before the addition of cells is done to re-hydrate and populate the scaffold with cells.

In another aspect of the present invention, the biodegradable hydrogel can include at least one attachment molecule to facilitate attachment of at least one cell thereto. The attachment molecule can include a polypeptide or small molecule, for example, and may be chemically immobilized onto the biodegradable hydrogel to facilitate cell attachment. Examples of attachment molecules can include fibronectin or a portion thereof, collagen or a portion thereof, polypeptides or proteins containing a peptide attachment sequence (e.g., arginine-glycine-aspartate sequence) (or other attachment sequence), enzymatically degradable peptide linkages, cell adhesion ligands, growth factors, degradable amino acid sequences, and/or protein-sequestering peptide sequences.

In some embodiments, the biodegradable hydrogel can have a macroporous structure that includes plurality of interconnected macropores that allow for easy encapsulation of cells as well as enhanced cell spreading and proliferation. The pores can have an average diameter of about 10 μm to about 800 μm (e. g., about 25 μm to about 100 μm).

The macropores can be generated in the biodegradable hydrogel by fabricating the hydrogel with a porogen that is capable of forming interconnected macropores in the hydrogel. The porogen can include any compound that will reserve a space within the hydrogel while the hydrogel is being formed and will diffuse, dissolve, and/or degrade prior to or after formation, implantation, or injection leaving a pore in the hydrogel.

Porogens may be of any shape or size. A porogen may be spheroidal, cuboidal, rectangular, elonganted, tubular, fibrous, disc-shaped, platelet-shaped, polygonal, etc. In certain embodiments, the porogen is granular with a diameter ranging from about 10 μm to about 800 μm (e.g., about 25 μm to about 100 μm). In certain embodiments, a porogen is elongated, tubular, or fibrous. Such porogens provide increased connectivity of pores of inventive composite and/or also allow for a lesser percentage of the porogen in the composite.

The amount of porogens may vary in the formation of the hydrogel and range from 1% to 80% by weight. In some embodiments, pores in hydrogels are thought to improve the osteoinductivity or osteoconductivity of the composite by providing holes for cells such as osteoblasts, osteoclasts, fibroblasts, cells of the osteoblast lineage, stem cells, etc. Pores provide inventive hydrogels with biological in growth capacity. Pores may also provide for easier degradation of the hydrogel. In some embodiments, a porogen is biocompatible.

In some embodiments, a porogen may be a gas, liquid, or solid. Exemplary gases that may act as porogens include carbon dioxide, nitrogen, argon, or air. Exemplary liquids include water, organic solvents, or biological fluids (e.g., blood, lymph, plasma). Gaseous or liquid porogen may diffuse out of the hydrogel before or after formation and/or implantation thereby providing pores for biological in-growth. Solid porogens may be crystalline or amorphous. Examples of possible solid porogens include water soluble compounds. Exemplary porogens include peptides and proteins (e.g., gelatin), carbohydrates, salts, sugar alcohols, natural polymers, synthetic polymers, and small molecules.

The concentration gradients can be physically formed within the hydrogel to facilitate release of one or more polynucleotides according to a gradient release profile. The gradient release profile can refer to the amount and/or rate of release of a polynucleotide from the degradable hydrogel. The gradient release profile can be selected for a particular hydrogel by modifying at least one property or characteristic (e.g., percentage of acrylation of the hydrogel forming polymers, concentration of polynucleotide, concentration of polynucleotide coupling polymeric molecules, number and type of hydrolysable or degradable bonds) of the material(s) used to form the hydrogel. Depending upon the modified property or characteristic, a different gradient will be formed and a different release profile will be produced. During formation of the biodegradable hydrogel, for example, the concentration of polynucleotide incorporated into the hydrogel can be increased or decreased to increase or decrease the concentration gradient of the polynucleotide upon release from the hydrogel.

The biodegradable hydrogel can be injectable and/or implantable, and can be in the form of a membrane, sponge, gel, solid scaffold, spun fiber, woven or unwoven mesh, nanoparticle, microparticle, or any other desirable configuration. The biodegradable hydrogel can be used in a variety of biomedical applications, including tissue engineering, drug discovery applications, and regenerative medicine and cancer therapy.

In one example of the present invention, a biodegradable hydrogel can be used to promote tissue growth in a subject. One step of the method can include identifying a target site. The target site can comprise a tissue defect (e.g., cartilage and/or bone defect) in which promotion of new tissue (e.g., cartilage and/or bone) is desired. The target site can also comprise a diseased location (e.g., tumor). Methods for identifying tissue defects and disease locations are known in the art and can include, for example, various imaging modalities, such as CT, MRI, and X-ray.

The tissue defect can include a defect caused by the destruction of bone or cartilage. For example, one type of cartilage defect can include a joint surface defect. Joint surface defects can be the result of a physical injury to one or more joints or, alternatively, a result of genetic or environmental factors. Most frequently, but not exclusively, such a defect will occur in the knee and will be caused by trauma, ligamentous instability, malalignment of the extremity, meniscectomy, failed ACI or mosaicplasty procedures, primary osteochondritis dessecans, osteoarthritis (early osteoarthritis or unicompartimental osteochondral defects), or tissue removal (e.g., due to cancer). Examples of bone defects can include any structural and/or functional skeletal abnormalities. Non-limiting examples of bone defects can include those associated with vertebral body or disc injury/destruction, spinal fusion, injured meniscus, avascular necrosis, cranio-facial repair/reconstruction (including dental repair/reconstruction), osteoarthritis, osteosclerosis, osteoporosis, implant fixation, trauma, and other inheritable or acquired bone disorders and diseases.

Where a tissue defect comprises a cartilage defect, the cartilage defect may also be referred to as an osteochondral defect when there is damage to articular cartilage and underlying (subchondral) bone. Usually, osteochondral defects appear on specific weight-bearing spots at the ends of the thighbone, shinbone, and the back of the kneecap. Cartilage defects in the context of the present invention should also be understood to comprise those conditions where surgical repair of cartilage is required, such as cosmetic surgery (e.g., nose, ear). Thus, cartilage defects can occur anywhere in the body where cartilage formation is disrupted, where cartilage is damaged or non-existent due to a genetic defect, where cartilage is important for the structure or functioning of an organ (e.g., structures such as menisci, the ear, the nose, the larynx, the trachea, the bronchi, structures of the heart valves, part of the costae, synchondroses, enthuses, etc.), and/or where cartilage is removed due to cancer, for example.

After identifying a target site, such as a cranio-facial cartilage defect of the nose, the biodegradable hydrogel can be administered to the target site. The hydrogel can be prepared according to the method described above.

Next, the biodegradable hydrogel may be loaded into a syringe or other similar device and injected or implanted into the tissue defect. Upon injection or implantation into the tissue defect, the biodegradable hydrogel can be formed into the shape of the tissue defect using tactile means.

After implanting the biodegradable hydrogel into the subject, the chondrocytes can begin to migrate from the hydrogel into the tissue defect, express growth and/or differentiation factors, and/or promote chondroprogenitor cell expansion and differentiation. Additionally, the presence of the biodegradable hydrogel in the tissue defect may promote migration of endogenous cells surrounding the tissue defect into the biodegradable hydrogel. Once implanted, the amide and/or ester linkages of the hydrolyzable covalent linkage between the base polymer and the cationic polymer/polynucleotide complex can be hydrolyzed. Hydrolysis of the covalent linkages can occur at a controlled rate and lead to controlled degradation of the biodegradable hydrogel. This hydrolytic degradation can create space for cell growth and deposition of a new extracellular matrix to replace the hydrogel.

The following examples is for the purpose of illustration only and is not intended to limit the scope of the claims, which are appended hereto.

### EXAMPLE 1

In this Example, a first hydrogel system was developed that would permit controlled, localized and sustained delivery of siRNA to cells incorporated within and surrounding the biomaterial. To achieve controlled siRNA release profiles, we designed photocrosslinked dextran (DEX) hydrogels covalently functionalized with cationic LPEI molecules via a biodegradable ester linkage. It was hypothesized that siRNA could be retained siRNA within the hydrogels via electrostatic interactions between the negatively charged siRNA and positively charged LPEI, and degradation of the ester linkages would permit tunable, controlled release of siRNA/LPEI complexes. The release profiles could be tailored by regulating the degree of these interactions and controlling the degradation rate of hydrogels. For this purpose, the hydrogels were photocrosslinked from solutions of DEX methacrylate containing various concentrations of methacrylated linear polyethyleneimine (LPEI). We tested whether LPEI modification affects the hydrogel physical properties, such as swelling, degradation profiles and mechanical properties, and the viability of human embryonic kidney 293 cells (HEK293) cultured near and within the hydrogels. Hydrogels containing varying DEX, LPEI and siRNA concentrations were examined to determine the role of these parameters on siRNA release profiles. Bioactivity of released siRNA and its ability to transfect cells inside the hydrogels were also investigated to demonstrate the utility of this system with tunable delivery profiles.

### Experimental

### Materials

DEX from Leuconostoc mesenteroides (average molecular weight of 40,000 g/mol), 4-(dimethylamino)pyridine (DMAP), glycidyl methacrylate (GMA, 97% pure), 2-hydroxyethyl methacrylate (HEMA), 1,1′-carbonyldiimidazole (CDI), dimethyl sulfoxide (DMSO), chloroform, deuterium oxide (D₂O) and Irgacure D-2959 were purchased from Sigma Aldrich (St. Louis, Mo., USA). Linear polyethyleneimine (LPEI, 25,000 g/mol) was purchased from Polysciences, Inc. (Warrington, Pa.). HEMA-IC was synthesized as previously reported [13]. CellTiter 96 Aqueous One Solution which contains 3-[4,5-dimethylthiazol-2-yl]-5-[3-carboxymethoxy-phenyl]-2-[4-sulfophenyl]-2H-tetrazolium (MTS-tetrazolium) was purchased from Promega Corp (Madison, Wis.). Dulbecco's modified eagle Medium with 4.5 g/L glucose (DMEM-HG) and fetal bovine serum (FBS) were purchased from Hyclone (Logan, Utah). Accell eGFP Control siRNA, Accell green cyclophilin B control and Accell Delivery Media (ADM serum-free) were obtained from Thermo Scientific Dharmacon (Lafayette, Colo.). Accell siRNA can enter cells without the use of a transfection reagent. HEK293 cells stably transfected with destabilized GFP (deGFP) were a generous gift from Piruz Nahreini, Ph.D. (University of Colorado Health Sciences Center). Falcon Transwell inserts were obtained from Becton Dickinson (Franklin Lakes, N.J.). Nuclease-free water was purchased from Ambion (Austin, Tex.). Dialysis membrane (MWCO 3500) was obtained from Spectrum Laboratories Inc. (Rancho Dominguez, Calif.).

### Synthesis of DEX Methacrylate and LPEI Methacrylate Macromers

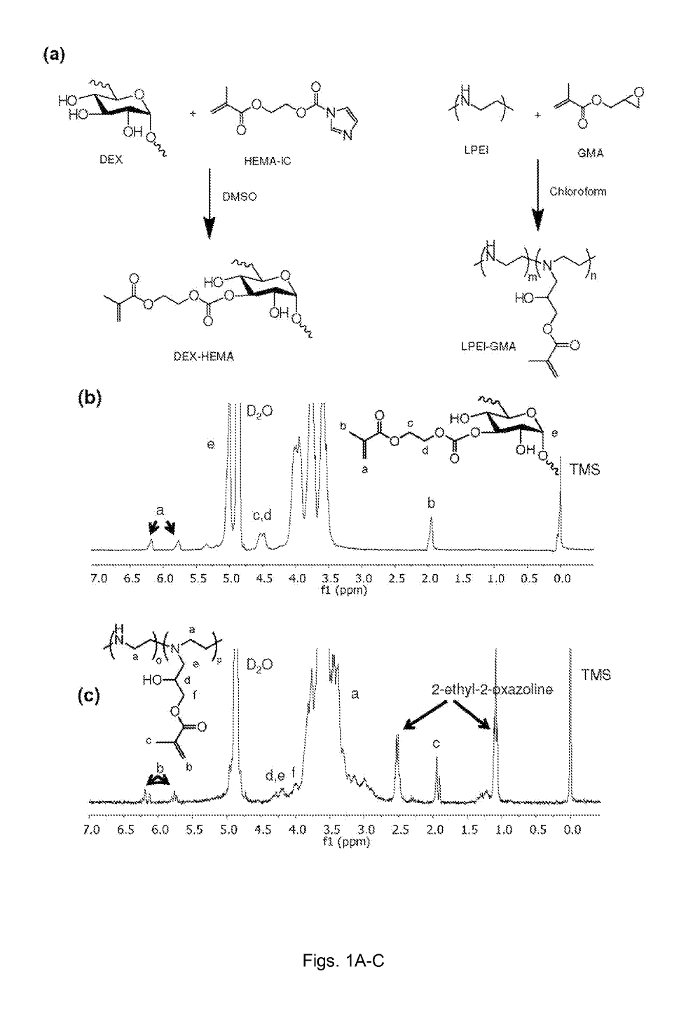

DEX methacrylate (DEX-HEMA) was synthesized via the reaction of HEMA-IC to the hydroxyl groups of the DEX main chain as previously described. Briefly, to synthesize DEX with 7% theoretical methacrylation, DEX (10 g) and DMAP (2 g) were dissolved in DMSO (90 ml) in a dry 250 ml round bottom flask. After complete dissolution, HEMA-IC (0.93 g) was added. The reaction occured for 4 days at room temperature, followed by dialysis (MWCO 3500) against ultrapure deionized water (diH₂O) for 3 days and lyophilization. White DEX-HEMA power (9 g) was obtained after lyophilization. LPEI methacrylate (LPEI-GMA) modified at a theoretical degree of 5% was synthesized via ring opening reaction of epoxy groups of GMA with amine groups of LPEI. LPEI (0.5 g) and GMA (105 μl) were dissolved in chloroform (30 ml) in a 250 ml round-bottom flask for 1 hour in a 60° C. silicon oil bath. The chloroform was then completely evaporated under vacuum and the mixture was reconstituted in ultrapure diH₂O (30 ml) at pH 6.0. The LPEI-GMA was purified by dialysis against ultrapure diH₂O at pH 6.0 (MWCO 3500) for 3 days, filtered using a 0.22 μm filter and lyophilized The final yield was 0.32 g. DEX-HEMA and LPEI-GMA were characterized by ¹H NMR in D₂O using a Varian Unity-300 (300 MHz) NMR spectrometer (Varian Inc., Palo Alto, Calif.). Peaks a and e in FIG. 1b and peaks b and the peaks from 2-ethyl-2-oxazoline in LPEI in FIG. 1c were used to determine the actual degree of modification of DEX-HEMA and LPEI-GMA, respectively.

### Photocrosslinking

DEX-HEMA (8 or 12% w/w) was dissolved in PBS with 0.05% w/v photoinitiator (Irgacure D-2959) and different LPEI-GMA concentrations (0, 5 and 10 μg/100 μl gel). The polymer solutions (100 μl) were placed into the wells of a 96-well plate and hydrogels were formed by photocrosslinking with 320-500 nm UV light at 20 mW/cm² for 85 sec using an Omnicure 51000 UV Spot Cure System (Lumen Dynamics Group, Mississauga, Ontario, Canada).

### Swelling and In Vitro Degradation

To determine the swelling profiles of these photocrosslinked DEX hydrogels, their dry and wet weights were determined at various time points over 17 days. The prepared photocrosslinked DEX hydrogels were lyophilized and their initial dry weights (W_{d} initial) were measured. Each dried hydrogel sample was immersed in 5 ml PBS at pH 7.4 and incubated at 37° C. The PBS was changed every three days. At predetermined time points, samples were removed and rinsed with diH₂O, and the weights of the swollen hydrogel samples (Wₛ) were measured. The swelling ratio (Q) was calculated by Q=Wₛ/W_{d} initial.

To determine the degradation profiles, each dried hydrogel sample was immersed in 5 ml of PBS and incubated at 37° C. The PBS was replaced every three days. At predetermined time points, samples were removed, rinsed with diH₂O and lyophilized The dry weights of the samples at different time points (W_{d}) were measured. The percent mass loss was calculated by (Wᵢ−W_{d})/Wᵢ×100, where Wi is the dry weight of the hydrogel samples at the initial time point. The samples (N=3 for each time point) were prepared and tested at the same time.

### Mechanical Properties

Rheological properties of photocrosslinked DEX hydrogels (12% w/w) with various LPEI concentrations with or without encapsulated siRNA (26.6 μg/100 μl hydrogel solution) were measured using a strain-controlled AR-2000ex rheometer (TA Instruments New Castle, Del.) with stainless-steel parallel plate geometry (plate diameter of 8 mm, gap of 0.75 mm) Hydrogels were made by pipetting solutions between two glass plates separated by two 0.75 mm spacers and photocrosslinked as previously described. Photocrosslinked hydrogel disks were punched out with an 8 mm biopsy punch to match the diameter of the parallel plates. G′ and G″ of each hydrogel were measured by performing a dynamic frequency sweep test in which a sinusoidal shear strain of constant peak amplitude (0.1%) was applied over a range of frequencies (0.1-10 Hz). The samples (N=3 per condition) were prepared and tested at the same time.

### Cell Viability

Photocrosslinked DEX hydrogels with various concentrations of LPEI were formed in cell culture inserts above a monolayer culture of HEK293 cells and cell viability was assessed using an MTS assay used according to manufacturer instructions. Briefly, HEK293 cells (passage 25) were seeded in a 24-well plate at 2×10⁵ cells/well in 0.5 ml of DMEM-HG containing 10% FBS and cultured at 37° C. and 5% CO₂ in a humidified incubator for 24 h. The DEX-HEMA solutions with various LPEI-GMA concentrations (100 μl, 12% w/w) were photocrosslinked in cell culture inserts (8 μm pore size), and then placed into the wells containing cells. Cells were cultured in parallel without the hydrogel or insert as a control. Media was changed every 3 days. After 2 and 7 days, media and inserts were removed, cells were rinsed with PBS, and a 20% CellTiter 96 Aqueous One Solution in PBS (0.5 ml) was added to each well. After 90 min incubation at 37° C., absorbance was measured at 490 nm on a plate reader (SAFIRE, Tecan, Austria). Cell viability was calculated by normalizing the absorbance of samples at 490 nm to that of the control.

To evaluate the viability of cells encapsulated in hydrogels of various LPEI concentrations, 2×10⁵ HEK293 cells (passage 27) were mixed into 100 μl of 12% w/w dextran solutions containing different LPEI concentrations. The cell/polymer mixtures were photocrosslinked in a 48-well plate and the cell-encapsutated hydrogels were cultured in 0.5 ml of DMEM-HG containing 10% FBS. The media was replaced every 3 days. After 7 days, media was removed and the hydrogels were rinsed with PBS. The cell-encapsulated hydrogels were homogenized for 30 sec using a TH homogenizer (Omni International, Kennesaw, Ga.) prior to measuring absorbances at 490 nm of a MTS assay as described above. The samples (N=3 per condition) were prepared and tested at the same time.

siRNA Release Kinetics

Accell green cyclophilin B control siRNA, which is fluorescently labeled with fluorescein isothiocyanate (FITC), was used to examine its release kinetics from DEX hydrogels (8 and 12% w/w). Hydrogels (8 and 12% w/w) with different methacrylated LPEI concentrations (0, 5, 10 μg), containing 13.3 or 26.6 μg of the siRNA, were fabricated in a 96-well plate. For example, to prepare hydrogels (12% w/w) with 13.3 μg of siRNA, first, 10 μl of siRNA (100 μM) was mixed with 17 μl of LPEI-GMA (pH 6.0), and the resulting mixture was allowed to sit for 30 min at room temperature. Second, 73 μl of DEX-HEMA solution (16.4% w/w) was added to the mixture. Then, the 100 μl mixture was pipetted into a 96-well plate, followed by exposure to UV light for 85 seconds. The formed hydrogel was transferred into a well of a 24-well plate containing 1 ml PBS. At predetermined time points, the PBS was removed and replaced. Separate standard curves were made for each condition by preparing matched hydrogels as described above and homogenizing in 1 ml PBS. The resulting solutions were then diluted to various concentrations (6, 2, 0.5, 0.05, 0 μg/ml). All the released siRNA and the standards were measured at pH 12 to dissociate the complexes using a plate reader (fmax, Molecular Devices, Inc., CA) set at excitation 485/emission 538. The samples (N=3 per condition) were prepared and tested at the same time.

Bioactivity of Released siRNA

100 μl hydrogel mixtures (12% w/w) were prepared as described above and then photocrosslinked in Transwell membranes (8 μm pore size) in 24-well plates. ADM (0.5 ml) was pipetted into the gel containing wells. The released siRNA in ADM was collected and refreshed at days 3, 7 and 14. HEK293 cells stably transfected with deGFP were seeded in 24-well plates at 1.5×10⁵ cells per well in media containing DMEM-HG and 10% FBS. After one day of incubation at 37° C. and 5% CO₂ in a humidified incubator, the media was removed and replaced with ADM containing the released siRNA from each time point above. The cells were collected and deGFP knockdown was determined by flow cytometry (EPICS XL-MCL, Beckman Coulter, Fullerton, Calif.) after two days of incubation. The samples (N=3 per condition) were prepared and tested at the same time.

### Transfection of Cells Encapsulated Inside Hydrogels

HEK293 cells were mixed with the hydrogel solutions (12% w/w) at a concentration of 20×10⁶ cells/ml. The cells/hydrogel solutions were transferred on to Transwell membrane inserts (0.4 μm pore size), and then crosslinked as described above. The cells/hydrogel constructs were initially cultured in ADM alone and the media was replaced with ADM+1% FBS at days 1, 3, 5, 7 and 9. Transfection was assessed using confocal microscopy (LSM510, Zeiss, Jena, Germany) on day 3, 7, and 11. Images were taken every 5 μm in the z-direction for 100 μm from the top of the hydrogels and compiled into a single 3-D projection. The samples (N=2 per condition) were prepared and tested at the same time.

### Statistical Analysis

All data are expressed as mean±standard deviation. Statistical significance was determined using Turkey-Kramer Multiple Comparisons Test with one-way analysis of variance (ANOVA) using InStat sofware (GraphPad Software, La Jolla, Calif.). p<0.05 was considered to be statistically significant.

### Results and Discussion

Synthesis and Characterization of the Polymer Macromers, and Hydrogel Formation

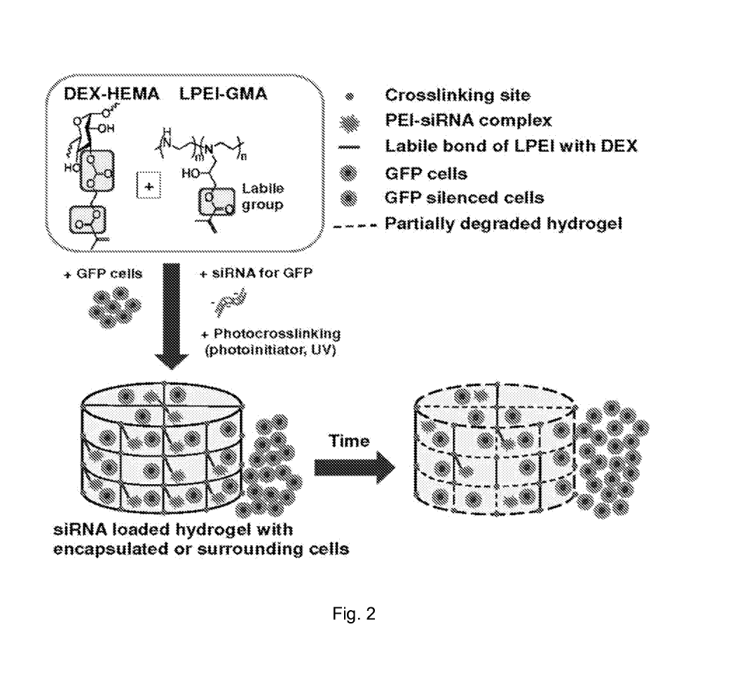

Methacrylated DEX (DEX-HEMA) and methacrylated LPEI (LPEI-GMA) were synthesized by coupling 2-hydroxylethyl methacrylate imidazolylcarbamate (HEMA-IC) to the hydroxyl groups of the DEX main chain and by the reaction of epoxy group of the GMA with amine groups of the LPEI, respectively, as shown in FIG. 1a. ¹H NMR spectra of the synthesized DEX-HEMA and LPEI-GMA in D₂O are presented in FIG. 1b and 1c. The signals corresponding to the methacrylate groups of both DEX-HEMA and LPEI-GMA appeared at 6.17 and 5.76 ppm, respectively. The peaks appearing at 4.48-4.55 ppm in FIG. 1b indicated the successful conjugation of HEMA-IC to DEX. Peaks at 4.0, 4.18 and 4.30 ppm in FIG. 1c confirm the ring opening reaction of epoxy group of GMA with amine groups of LPEI. From the ¹H NMR spectra, the actual degrees of methacrylation of DEX and LPEI were 4.2% and 1.2%, respectively. DEX-HEMA macromers were photocrosslinked with various concentrations of LPEI-GMA macromers to form hydrogels for controlled, localized and sustained delivery of siRNA to inhibit gene expression of surrounding or encapsulated cells (FIG. 2).

### Swelling Kinetics and Degradation of the Photocrosslinked Hydrogels

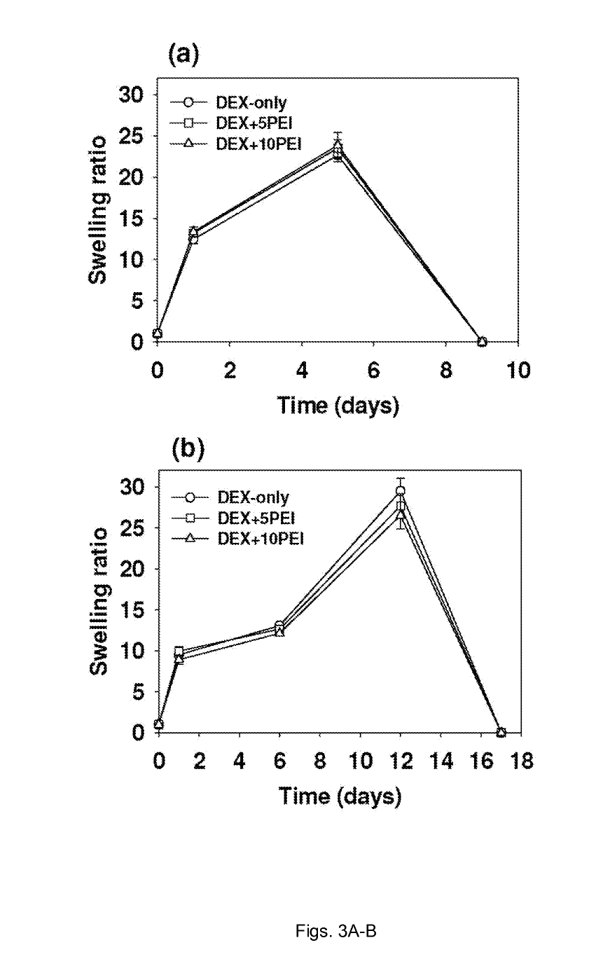

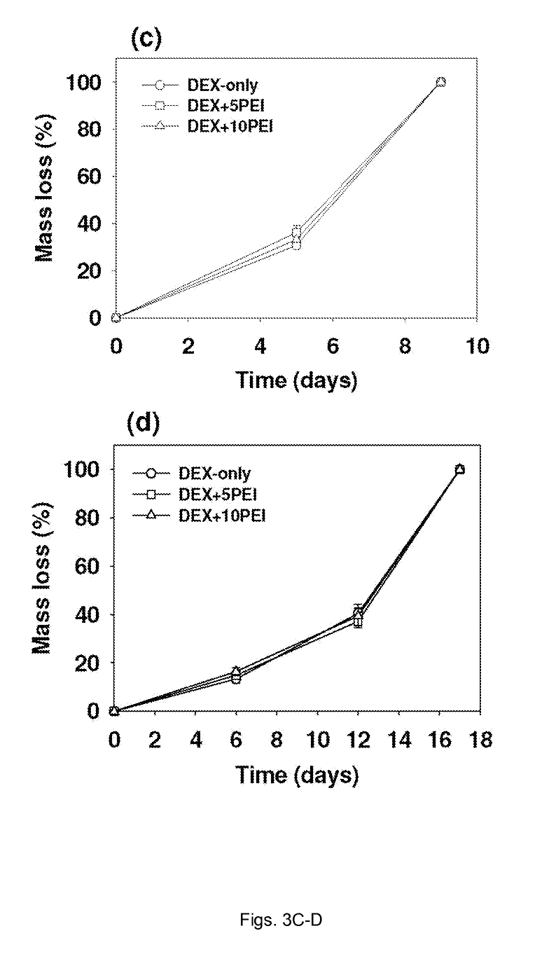

A photocrosslinked hydrogel system was formed by combining neutral DEX-HEMA and cationic LPEI-GMA macromers capable of forming electrostatic interactions with siRNA. DEX-HEMA, previously reported by Hennink, is a biodegradable and biocompatible polymer, the covalent crosslinks formed following photopolymerization under low level UV light contain ester linkages that can degrade in aqueous media. Biomaterial swelling and degradation rate are important for (1) transport of oxygen and nutrients to and removal of waste products from incorporated cells, (2) providing space for new tissue formation in tissue engineering applications, and (3) controlling the release of bioactive molecules such as siRNA. These parameters can be tailored by varying the degree of methacrylation, size of crosslinker, and hydrogel macromer concentration. Swelling ratios of photocrosslinked DEX hydrogels (8 and 12% w/w) with 0, 5 and 10 μg LPEI/100 μL gel (DEX-only, DEX+5PEI and DEX+10PEI, respectively) in phosphate buffered saline (PBS) over the course of 9 and 17 days are shown in FIGS. 3a & 3b. The DEX hydrogels with various LPEI concentrations displayed similar swelling profiles, indicating that the LPEI did not affect this hydrogel property at the concentrations investigated. The 8% w/w DEX hydrogels achieved maximal swelling after 5 days, whereas the 12% w/w DEX hydrogels reached maximal swelling after 12 days.

Mass loss (%) of photocrosslinked DEX hydrogels with various LPEI concentrations was examined by measuring their dry weights over time (FIGS. 3c & 3d). The 8% w/w DEX hydrogels were completely degraded by day 9, while the 12% w/w DEX hydrogels were not completely degraded until day 17 due to the higher crosslinking density of the hydrogels formed with higher macromer concentration. At both concentrations of DEX in the hydrogels, there was no significant difference in degradation profiles with various LPEI contents.

### Rheology

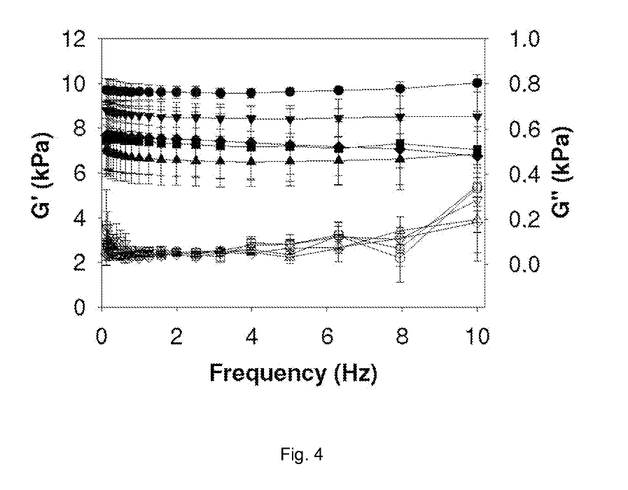

To investigate whether the addition of LPEI or siRNA alters the mechanical properties of the hydrogels, storage (G′) and loss (G″) moduli were measured using a rheometer. G′ and G″ represent the elastic and viscous behavior, respectively, of the hydrogels. FIG. 4 shows G′ and G″ of the DEX hydrogels (12% w/w) with different LPEI contents with and without incorporated siRNA. G′ of the DEX+5PEI and DEX+10PEI hydrogels was slightly greater than that of the DEX-only hydrogel. Hydrogels with siRNA exhibited a trend towards higher G′ than those without siRNA. In addition, G′ was greater than G″ for all frequencies tested (0.1-10 Hz), indicating that elastic behavior of the hydrogels dominates in this range. The increased hydrogel G′ with LPEI likely resulted from increased crosslinking density of the hydrogels, and the increase in G′ with siRNA may be a result of higher density of the hydrogel constructs.

### Cell Viability

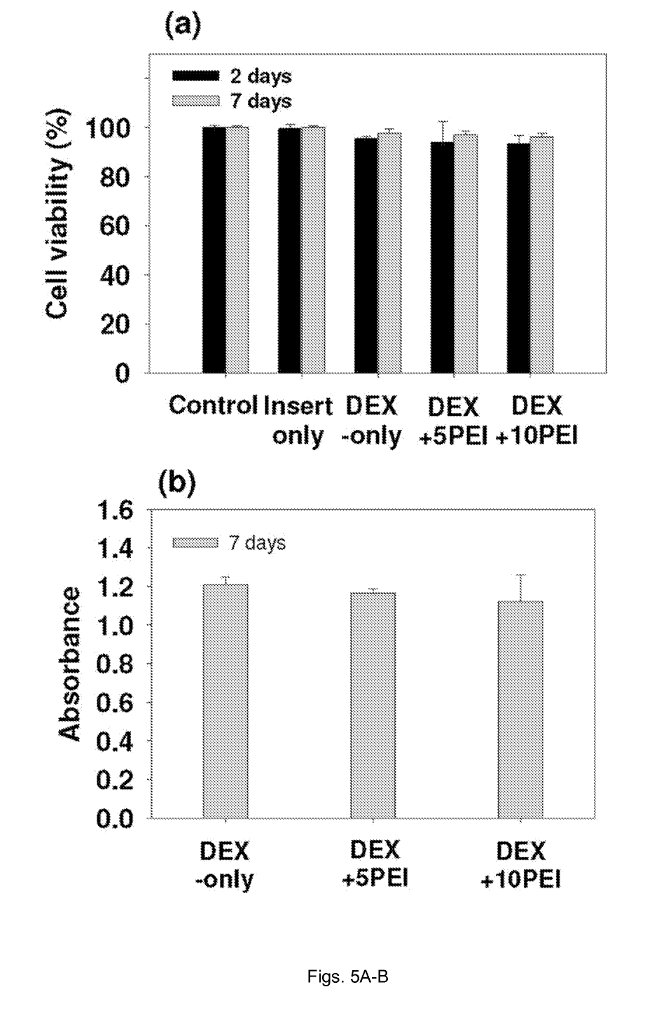

Hydrogels for use in biological molecule delivery or tissue engineering applications must be cytocompatible. LPEI, a cationic synthetic polymer, was modified with GMA to make it photocrosslinkable with DEX-HEMA. Since LPEI can be cytotoxic to cells, we tested the viability of deGFP expressing HEK293 cells cultured in the presence of photocrosslinked hydrogel degradation products. These cells were used because they constitutively express deGFP, which gets turned off by the released siRNA in our bioactivity evaluation experiments. Photocrosslinked DEX hydrogels (12% w/w) with different LPEI concentrations were prepared in cell culture inserts above a monolayer culture of HEK293 cells and cell viability was determined using an MTS assay, which measures mitochondrial metabolic activity, and normalized to wells containing cells and culture medium only (FIG. 5a). After 2 days in culture, cell viability in the presence of DEX hydrogels with various LPEI concentrations (DEX-only, DEX+5PEI and DEX+10PEI) was 95.42%±0.96, 94.09%±8.44 and 93.56%±3.03, respectively, that of the control wells. After 7 days in culture, they remained highly viable at 97.63%±1.73, 97.00%±1.70 and 96.00%±1.52, respectively, compared to the controls. No significant differences were found between experimental conditions and the control or between time points.

Cell viability of HEK293 cells encapsulated within 12% w/w DEX hydrogels containing various LPEI concentrations was also determined by the MTS assay to examine survival of the cells during photocrosslinking formation of the hydrogels and in 3D culture (FIG. 5b). Since photocrosslinked hydrogels formed with DEX-HEMA have been previously shown to be cytocompatible, we compared MTS assay absorbances at 490 nm of cells in the DEX+5PEI and DEX+10PEI hydrogels to that of cells in the DEX-only control. There was no significant difference between the absorbances of the samples (1.21±0.04 for DEX-only, 1.165±0.022 for DEX+5PEI, and 1.124±0.135 for DEX+10PEI) after 7 days in culture, which indicates that the addition of LPEI-GMA to the system did not affect cell viability. While in monolayer culture HEK293 cells may require cell adhesion for survival, this is not the case in 3D culture as our group and others have shown that HEK293 cells remain viable when cultured within hydrogels that do not promote cell adhesion such as alginate.

siRNA Release

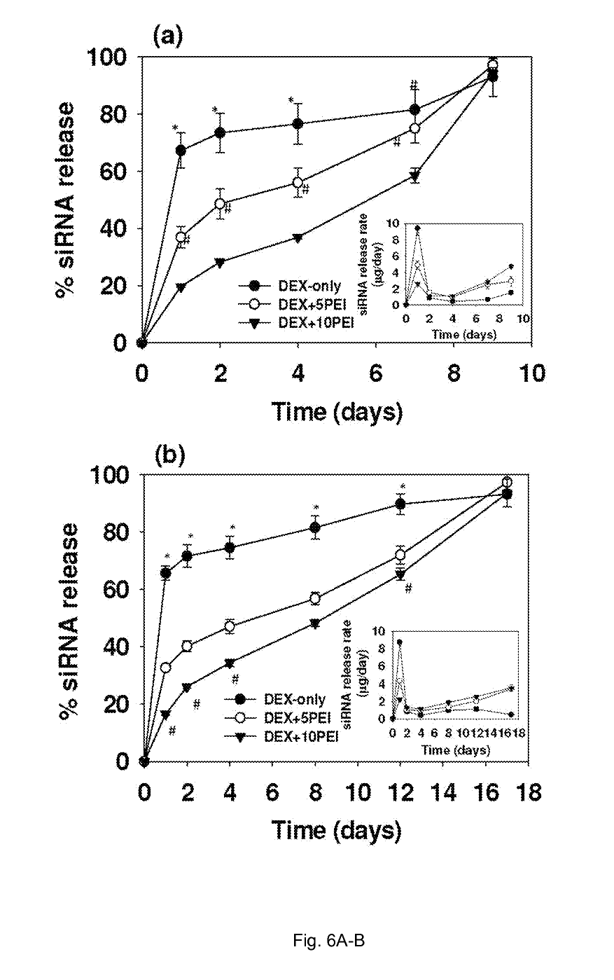

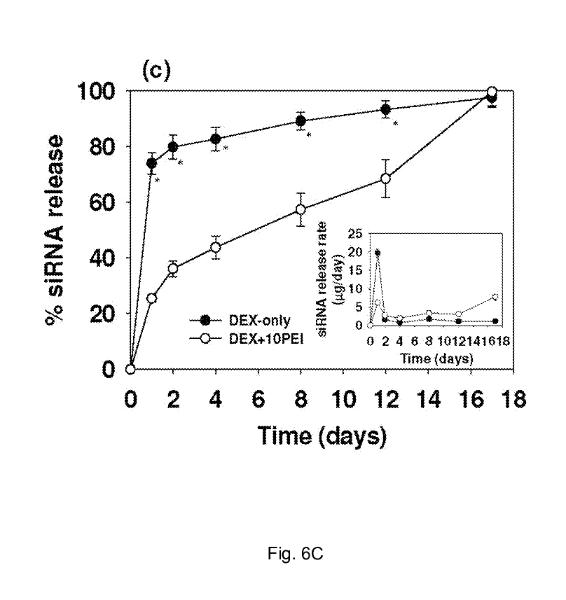

PEI is capable of complexing with and condensing siRNA into nanoparticles which can protect siRNA from denaturation by ribonucleases and enhance cellular uptake. For example, siRNA/PEI complexes have been reported to silence VEGF expression to reduce tumor growth and siRNA/PEI conjugates physically trapped in scaffolds were released to suppress fibroblast proliferation and knockdown type 1 collagen mRNA expression. In this study, LPEI was utilized because of two of its important properties: 1) its capacity to be chemically modified via amine groups on its backbone and 2) its ability to form stabilized complexes with siRNA. To examine whether the covalent incorporation of LPEI into the DEX hydrogels during photopolymerization could delay and control the release of siRNA over a prolonged time period, the temporal release of siRNA from 8 and 12% w/w hydrogels with various LPEI contents was compared (FIG. 6). The release was sustained and delayed as the LPEI amount was increased from 5 to 10 μg. For example, on the first day, the cumulative release of siRNA from the DEX-only hydrogels (8 and 12% w/w) with siRNA loading amount of 13.3 μg was 67.22% and 65.59% whereas the release from the DEX+5PEI hydrogels was significantly less at only 36.92% and 32.61%, respectively (FIGS. 6a & 6b). Additionally, even less siRNA (19.63% and 16.54%) was released from the DEX+10PEI hydrogels (8 and 12% w/w, respectively) on the first day. With siRNA loading amount of 26.6 μg, a similar trend was observed at the first day (FIG. 6c). These results show that the significant initial burst release resulting from the hydrogels without LPEI (DEX-only) decreased markedly by the covalent conjugation of a small amount of LPEI (DEX+5PEI and DEX+10PEI). Insets show siRNA release rate (μg/day) from the corresponding hydrogels.

The release was also controlled by the hydrogel concentration. For example, the 8% w/w hydrogels released the siRNA over the course of 9 days as the hydrogels degraded completely (FIG. 6a). Similarly, siRNA was released from the 12% w/w hydrogels until they completely degraded by day 17 (FIG. 6b). siRNA was released from the DEX-only hydrogels predominantly via simple diffusion in addition to the degradation of the hydrogels at later time points, resulting in a substantial initial burst release. The addition of free PEI into hydrogels does not allow for control over the release because the siRNA/PEI complexes simply diffuse out of the hydrogels. Therefore, to achieve control over the release, LPEI was modified with GMA which could covalently bind to the hydrogels via free radical photopolymerization and form hydrolysable ester-containing crosslinks Importantly, the interactions were independently controlled by adding defined amounts of LPEI-GMA and siRNA prior to photocrosslinking gelation. The mechanism of siRNA release from the LPEI-containing hydrogels at later time points is thus regulated predominantly by controlled degradation of the hydrogels and the ester linkages between the LPEI and DEX, followed by diffusion of the siRNA/LPEI complexes from the hydrogels.

### Bioactivity Evaluation

This study would determine whether the UV light used in the photocrosslinking process, the photoinitiator, the electrostatic interactions of siRNA and methacrylated LPEI, and the subsequent release of siRNA from the hydrogels affects the gene silencing ability of the released siRNA or siRNA/LPEI complexes. siRNA against deGFP (13.3 and 26.6 μg/gel) was released in ADM from 12% w/w DEX hydrogels (DEX-only, DEX+5PEI and DEX+10PEI), which degraded completely after 14 days of culture, and collected at days 3, 7 and 14. HEK293 cells plated in tissue culture plastic one day prior were then exposed to collected siRNA release samples. deGFP expression of these cells was measured by flow cytometry after 48 h treatment with the released siRNA.

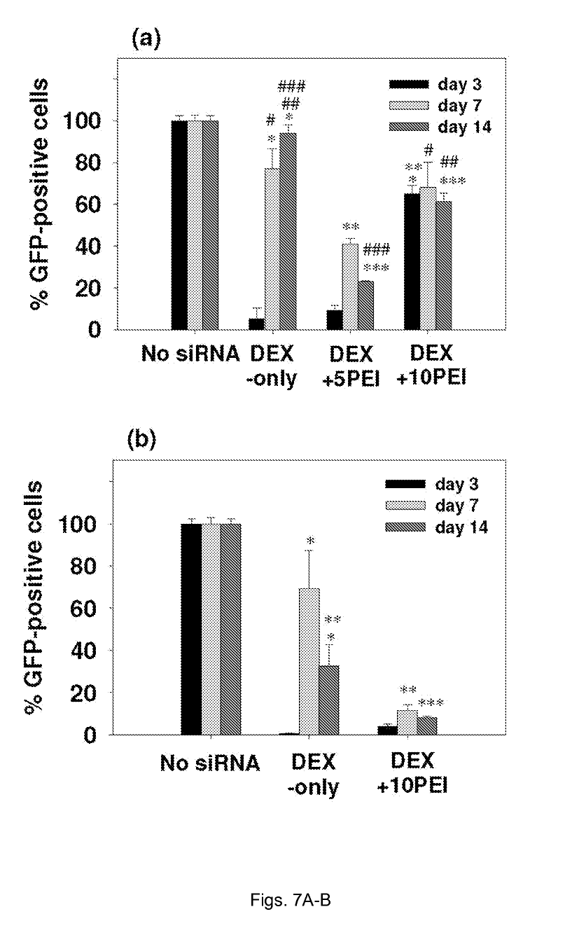

Hydrogels (12% w/w) displayed a sustained silencing of deGFP expression only when LPEI was covalently incorporated within the hydrogels (FIGS. 7a & 7b). deGFP expression of cells exposed to siRNA (13.3 μg original mass) released from DEX-only hydrogels was silenced to 5.23% of control samples with releasates from day 3, but increased to 77.10% and 94.13% with releasates from days 7 and 14, respectively (FIG. 7a). In contrast, the percentage of deGFP-positive cells treated with siRNA released from DEX+5PEI hydrogels was 9.71, 40.89 and 23.16% with releasates from days 3, 7 and 14, respectively, and ˜70% for all three time points with siRNA from DEX+10PEI hydrogels. These results indicate more sustained knockdown when LPEI was coupled to the hydrogels. When siRNA amount in the hydrogels was increased to 26.6 μg, deGFP knockdown increased (FIG. 7b). Importantly, deGFP expressing cells were reduced to less than 11.56% for releasates from all time points from the DEX+10PEI hydrogels. These results confirm the siRNA and siRNA/LPEI complexes released from the hydrogels retained their capacity to substantially knockdown deGFP expression in HEK293 cells cultured on tissue culture plastic. The magnitude and duration of knockdown was dependent on the amount of siRNA and coupled LPEI present in the hydrogels.

Transfection of Cells Incorporated into Hydrogels

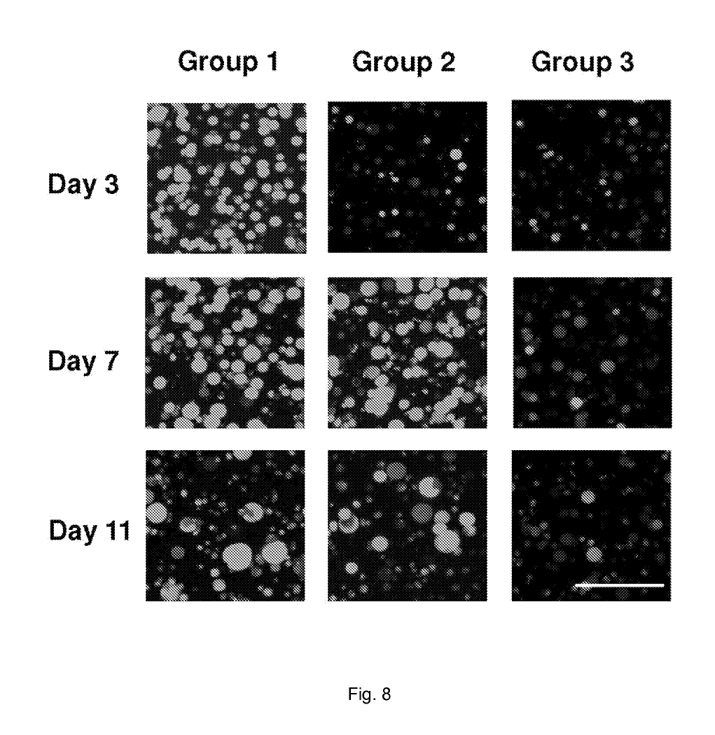

Hydrophilic, biocompatible, and biodegradable 3D hydrogel networks, can serve as temporary matrices for cell growth and new tissue formation in regenerative medicine applications when implanted or injected at a defect or damaged tissue site. Therapeutics incorporated into hydrogels, can be retained and then locally released to transplanted cells within the hydrogels and to host cells at specific sites to treat diseases or promote healing of damaged tissues. Therefore, we examined the ability of this system to knockdown deGFP expression of cells cultured within the hydrogels. Three groups of 12% w/w DEX hydrogels were used: 1) DEX-only hydrogels without siRNA (control); 2) DEX-only hydrogels with 26.6 μg siRNA; 3) DEX+10PEI hydrogels with 26.6 μg siRNA. Photomicrographs depicting the fluorescence activity of cells in the hydrogels as a measure of transfection are shown in FIG. 8. deGFP expression of cells in the hydrogels with siRNA (groups 2 and 3) was substantially reduced at day 3, whereas that of cells in the hydrogels without siRNA (group 1) was not. Compared to strong deGFP expression of cells in groups 1 and 2 at time points of 7 and 11 days, cells in group 3 exhibited substantial deGFP silencing even at these later time points. This demonstrates that the coupled LPEI in group 3 was acting to retain bioactive siRNA within the hydrogels for a longer period of time to affect incorporated cells.

Cells in group 1 strongly expressed deGFP at all time points because they were not transfected with siRNA (FIG. 8). deGFP expression of cells in group 2 that were transfected with siRNA was significantly reduced at day 3, but increased at days 7 and 11 (FIG. 8) since most of the siRNA had been released from the hydrogels by these time points (FIG. 6c). In contrast, cells in group 3, which were encapsulated in hydrogels containing LPEI which acted to retard siRNA release, exhibited sustained deGFP knockdown at days 3, 7 and 11 in culture. Due to the swelling of the hydrogels, the density of cells imaged on the confocal microscope on day 11 in all groups decreased for all conditions. At later time points, large areas of green represent cell clusters that arise due to cell proliferation and merging. Their morphology is round as would be expected within a material that does not support cell adhesion. We previously reported photocrosslinked alginate hydrogels without coupled LPEI that silenced deGFP expression of incorporated cells at day 3, but at a later time point (day 6) deGFP expression increased significantly. This result is similar to our current findings with cells incorporated into hydrogels in group 2. In group 2, siRNA was released from the hydrogel by simple diffusion or degradation. In contrast, in group 3 the release of siRNA was governed by a small amount of covalently coupled LPEI molecules, resulting in a subsequent extended deGFP knockdown.

### Example 2

In this Example we describe engineered in situ forming poly(ethylene glycol) (PEG) hydrogels that provided a platform for tunable, controlled and local release of siRNA to promote osteogenic differentiation of encapsulated human MSCs (hMSCs). The hydrogels could form by simple mixing two macromer components at physiological conditions without the need of photoinitiators, chemical or UV exposure that may be harmful to incorporated cells or bioactive factors.

### Hydrogel Formation

Hydrogels were fabricated by mixing 8-arm-PEG-MAES or 8-arm-PEG-A and 8-arm-PEG-SH (10,000 g/mol, JenKem Technology USA, Allen, Tex.) solutions in DPBS (pH 7.4, Fisher Scientific, Pittsburgh, Pa.) with a 1:1 stoichiometry ratio of acrylate and thiol groups to obtain a final concentration of 15% w/v. After mixing, the hydrogel solutions (100 μl) were immediately placed into a 15 ml conical tube and allowed to gel at 37° C. Hydrogels formed within two minutes, but they were incubated for further 2 h to achieve maximum gelation. Gelation time of the hydrogels is examined via the visual tube inversion method in PBS at pH 7.4 and room temperature. Specifically, each hydrogel solution (100 μl) was added in a microcentrifuge tube, vortexing for 10 sec and gelation time was then monitored. The gelation time is determined when the solutions stop flowing.

siRNA Release

siRNA fluorescently tagged with fluorescien isothiocyanate (FITC), Green cyclophilin B control siRNA (Thermo Scientific Dharmacon, Lafayette, Colo.), was used to examine its release kinetics from PEG hydrogels. siRNA was complexed with PEI (25,000 g/mol, Sigma, St. Louis, Mo.) in DPBS at pH 7.4 with an N/P ratio of 10 to form polyplexes that were then encapsulated within the hydrogels prepared as mentioned above. 4 μg siRNA was used for each 100 μl hydrogel. Each hydrogel loaded with siRNA/PEI nanocomplexes were placed into a 15 ml conical tube containing 1 ml DPBS. The release was carried out at 37° C. and the DPBS was taken out and replaced with a fresh 1 ml DPBS at given time points. Standard curves were prepared using siRNA/PEI nanocomplexes as described above. The siRNA samples were measured in IN NaOH solutions to dissociate the complexes using a plate reader (fmax, Molecular Devices, Sunnyvale, Calif.) set at excitation 485/emission 538. Three hydrogels per condition (N=3) were prepared for at each time point.

### Bioactivity Evaluation

For the bioactivity evaluation, siGFP and siLuc (Thermo Scientific Dharmacon) were complexed with PEI as described above and the siRNA/PEI complexes were released from the M, MA and A hydrogels. The siRNA/PEI complexes released during the last five day when the gels degraded completely were used for bioactivity evaluation. One day before transfection, deGFp expressing HEK293 cells (passage 27, a generous gift from Piruz Nahreini, PhD., University of Colorado Health Sciences Center) were plated into 24-well plates (Fisher Scientific) at a density of 100,000 cells per well and incubated for 24 h at 37° C. and 5% CO₂. At the day of transfection, the siRNA/PEI samples (0.26 μg siRNA) were treated with HEK293 cells plated in monolayers in tissue culture plastics one day earlier. The complexes were incubated with the cells for 6 h and then the media were replaced with Dulbecco's Modified Eagle Medium High Glucose (DMEM-HG, HyClone, Logan, Utah) with 10% fetal bovine serum characterized (FBS, HyClone). The cells were harvested in DPBS for GFP knockdown quantification using flow cytometry (EPICS XLMCL, Beckman Coulter, Fullerton, Calif.) after cultured two days at 37° C. and 5% CO₂. Freshly prepared siLuc/PEI and released siLuc/PEI complexes were used as negative controls. Bioactivity of the released siGFP/PEI complexes was compared to the freshly prepared siGFP/PEI complexes. The results are normalized to the cells without siRNA treatment and the experiments are performed in triplicate (N=3).

Osteogenic Differentiation of hMSCs Encapsulated in Hydrogels

hMSCs were isolated from the posterior iliac crest of healthy donors under a protocol approved by the University Hospitalsof Cleveland Institutional Review Board and processed by the Skeletal Research Center Mesenchymal Stem Cell Core Facility as previously described. Briefly, the aspirates were washed with growth medium comprised of Low Glucose Dulbecco's Modified Eagle's Medium (DMEM-LG, Sigma) with 10% prescreened FBS. Mononucleated cells were isolated by centrifugation in a Percol (Sigma) density gradient and the isolated cells were plated at 1.8×10⁵ cells/cm² in growth medium. Medium was changed every 3 days and after 14 days of culture the cells were passaged at a density of 5×10³ cells/cm². hMSCs (passage 3) were suspended in hydrogel solutions containing siNoggin (Insight Genomics, Falls Church, Va.) or/and miRNA-20a (Insight Genomics) complexes at a density of 5×10⁶ cells/ml. Hydrogels (100 ul) were formed in microcentrifuge tubes as described above, and then each gel was transferred into each well of 24 well plates containing 1 ml osteogenic media (10 mM β-glycerophosphate (CalBiochem, Billerica, Mass.), 50 μM ascorbic acid (Wako USA, Richmond, Va.), 100 nM dexamethasone (MP Biomedicals, Solon, Ohio) and 100 ng/ml bone morphogenetic protein-2 (GenScript, Piscataway, N.J.). The osteogenic media were changed two times a week. At predetermined time point, each hydrogel-cell construct was removed from the 24-well plates, put in 1 ml ALP lysis buffer and homogenized at 35,000 rpm for 30 s using a TH homogenizer (Omni International, Marietta, Ga.). The homogenized solutions were centrifuged at 500 g with a Sorvall Legent RT Plus Centrifuge (Thermo Fisher Scientific). The supernatants were collected for ALP, calcium and DNA analysis (N=3).

For ALP measurement, the supernatant (100 μl) was treated with ALP substrate with p-nitrophenylphosphate (pNPP, 100 μl, Sigma), and then 0.1 N NaOH (50 μl) was added to stop the reaction. The absorbance was measured at 405 nm using a plate reader (VersaMax, Molecular Devices, Sunnyvale, Calif.). Calcium content of the encapsulated hMSCs was also quantified using calcium assay kit (Pointe Scientific, Canton, Mich.) according to the company's instruction. The supernatant (4 μl) was mixed with a color and buffer reagent mixture (250 μl) and the absorbance was read at 570 nm on a VersaMax plate reader. DNA was also measured using a Picogreen assay kit (Invitrogen) on a plate reader (fmax, Molecular Devices) set at excitation 485/emission 538. All the ALP and calcium contents were normalized to DNA content. Calcium deposition in the bulk gels was stained with Alizarin red (Sigma).

RNA Isolation and Real-Time Quantitative Reverse Transcription-Polymerase Chain Reaction (qRT-PCR)

RNA was isolated from encapsulated hMSCs on day 7, 14 and 28 using TRI reagent (Sigma). cDNA was prepared using Superscript III reverse transcriptase (Invitrogen, Carlsbad, Calif.) according to the manufacturer's instruction, and then was used for qRT-PCR analysis with SYBR Green Master Mix (Applied Biosystems, Foster City, Calif.). The reactions were performed on an ABI 7700 Real-Time PCR instrument (Applied Biosystems). Expression levels of noggin, ALP, runx2, BSP and PPAR-y were examined by qRT-PCR at days 7, 14 and 28.

### Results

In situ forming hydrogels were formed via Michael type reaction of acrylated eight-arm-PEG and thiolated eight-arm-PEG (8-arm-PEG-SH) in PBS pH 7.4 with a 1:1 stoichiometric ratio of acrylate and thiol. The acrylated 8-arm-PEGs were 8-arm-PEG-mono(2-acryloyloxyethyl) succinate (8-arm-PEG-MAES) and 8-arm-PEG-acrylate (8-arm-PEG-A). While there were three ester groups on each arm of 8-arm-PEG-MAES, each arm of 8-arm-PEG-A contained one ester bond. We expected the hydrogels resulting from 8-arm-PEG-MAES would hydrolytically degrade more rapidly than those from 8-arm-PEG-A, due to their greater ester bond density in each macromer molecule. 8-arm-PEG-MAES was synthesized via the esterification reaction of the hydroxyl groups of 8-arm-PEG and the carboxylic acid of MAES in the presence of 4-(dimethylamino)pyridinium 4-toluenesulfonate (DPTS) as a catalyst. 8-arm-PEG-acrylate (8-arm-PEG-A) was prepared by the reaction of the hydroxyl groups of PEG with acryloyl chloride, as previously reported. While gels M and A were composed of 8-arm-PEG-MAES and 8-arm-PEG-A, respectively, with 8-arm-PEG-SH, gel MA was a mixture of 8-arm-PEG-MAES and 8-arm-PEG-A (weight ratio 2/1) with 8-arm-PEG-SH. Gelation rates, observed via a tube inversion method in PBS at pH 7.4, for the gels M, MA and A were 62.66±2.51, 59.00±3.60 and 53.00±2.64 sec, respectively,

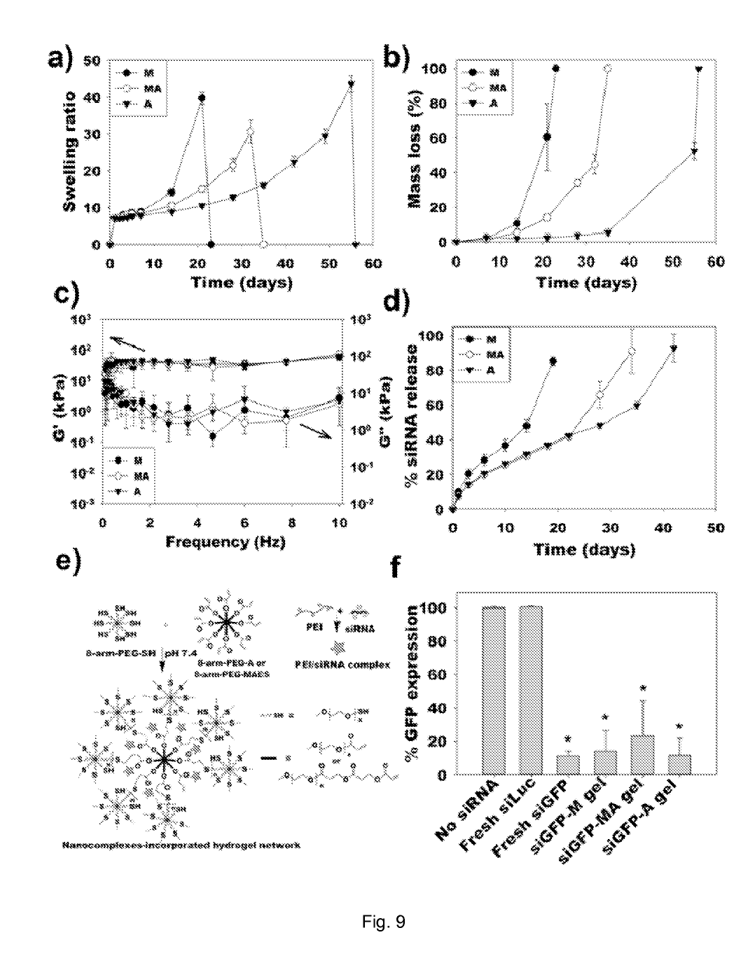

Physical and chemical structures of the hydrogels were examined though swelling, degradation and rheology measurements. Swelling ratio and hydrolytic degradation rates were faster for the M, then MA and finally A gels (FIGS. 9a and 9b), likely due to the more hydrolytically degradable ester density in the gels M, MA and A, respectively. The gel M degraded slowly for first two weeks before its hydrolytic degradation kinetics increased rapidly by week 3. Similarly, the gel MA degraded gradually and then the degradation accelerated after 3 weeks. Interestingly, mass loss of the gel A degraded slowly for first 5 weeks before it increased rapidly over time. In addition, mechanical strength of the hydrogels was also been examined with a rheometer. Storage (G′) and (G″) loss moduli that represent elastic and viscous properties, respectively, of the hydrogels are shown in FIG. 9c. G′ of the three hydrogels were similar and higher than G″ in the whole range of tested frequency (0.1-10 Hz), indicating the elasticity of the hydrogels were dominant.

To assess the ability of the biomaterial system to retain siRNA, release of siRNA/polyethyleneimine (PEI) complexes from the three hydrogels over time was performed in PBS at 37° C. (FIG. 9d). The complexes were prepared separately and homogeneously mixed with hydrogel macromers for their incorporation prior to gelation (FIG. 9e). The release rate of the complexes from the gel M was greater than that from the gels MA and A. Most of siRNA was released from the M, MA and A gels after 22, 35 and 42 days, respectively, without an initial burst release. The results indicate that these hydrogels could slowly release siRNA in a sustained way over a prolonged period of time.

To assure that hydrogels can protect and preserve bioactivity of siRNA over the entire release time, siRNA targeting GFP and luciferase genes (siGFP and siLuc, respectively) were complexed with PEI and then the complexes were incorporated into the hydrogels. The siRNA/PEI complexes released during the last five days when the gels degraded completely were exploited to assess their capability to silence GFP expression in HEK293 cells. Cells were treated with equal amounts of the released complexes. Percent GFP knockdown was normalized to the untreated cells in media only (controls), and compared to the freshly prepared siGFP and siLuc complexes. As shown FIG. 9f, while % GFP expression of the untreated cells remained 100%, the cells treated with the fresh or released siGFP/PEI significantly reduced their GFP expression to around 10-20%. siLuc, a non-targeting control, did not reduce GFP expression in comparison to the untreated cell group. GFP expression of cells transfected with the released siLuc remained 100% compared to the control. The results indicate that bioactivity of the released siRNA that was in the complex form was preserved and it was not significantly different from freshly prepared siGFP/PEI complexes.

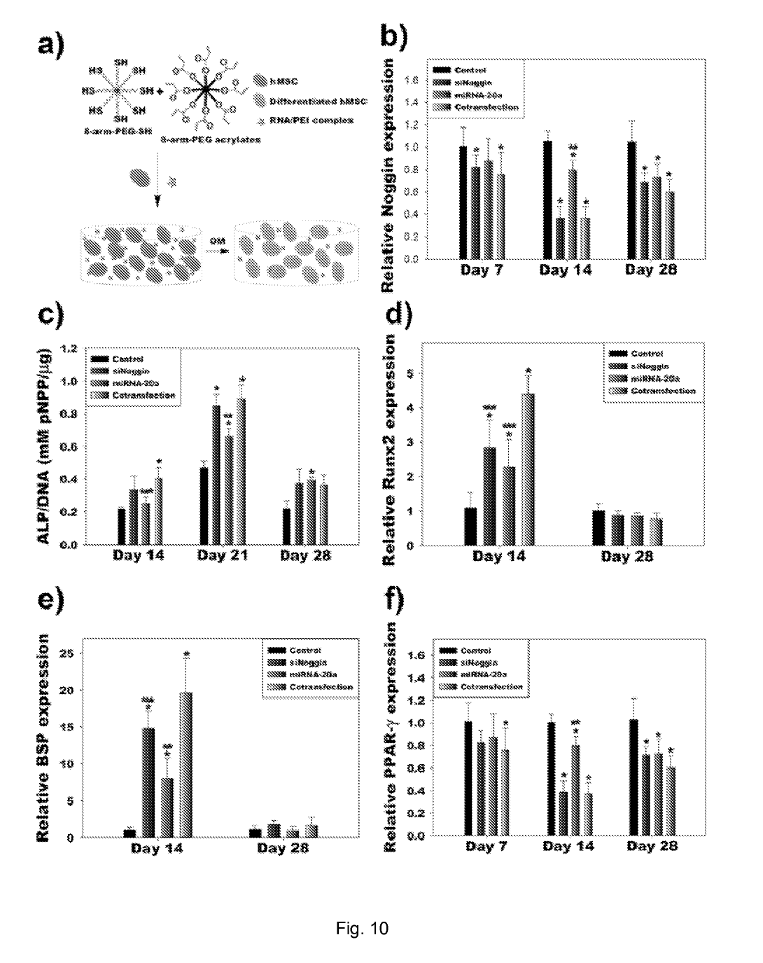

We have demonstrated above that the biomaterials could retain siRNA within them over a prolonged period of time and the released siRNA was still able to knockdown GFP as a marker for gene expression. We then examined whether the RNA/hydrogel constructs could support osteogenic differentiation of hMSCs encapsulated in the hydrogels. Recently, gene silencing of osteogenic suppressors such as noggin and chordin, has been studied to enhance osteogenic differentiation in stem cells derived from bone marrow or adipose tissue with increased osteogenic gene expression and mineralization. In addition, miRNA-20a transfection showed increased osteogenic response of hMSCs with acceleration of osteogenic maker expression and mineralization. In this study, we have delivered siNoggin and miRNA-20a separately or in combination in a controlled, sustained manner from in situ PEG hydrogel (A gel) to accelerate the osteogenic differentiation of encapsulated hMSCs and mineralization by silence Noggin and/or miRNA expression (FIG. 10a). Viability of hMSCs encapsulated in the biomaterials with RNAs remained high, indicating that the hydrogels were cytocompatible.

Since the noggin expression is upregulated by BMP-2 treatment during osteogenic differentiation of cells, a sustained gene suppression of noggin may be important in bone regeneration by osteogenic differentiation of transplanted hMSCs with BMP-2 delivery. Therefore, quantitative mRNA expression analysis of noggin was performed at day 7, 14, and 28 to determine the subsequent noggin suppression (FIG. 10b). hMSCs encapsulated with either siNoggin or siNoggin and miRNA-20a exhibited significant gene silencing by 28 day within 3D microenvironment as compared to control group (hMSCs in gels with negative control RNA). Since noggin has been shown to have negative effect on osteogenic differentiation of stem cell, this injectable in situ PEG hydrogel RNA delivery systems is promising to provide great utility for osteogenic differentiation of stem cells for bone tissue engineering by localized and subsequent noggin silencing to encapsulated stem cells.

To investigate the effect of localized, sustained delivery of siNoggin and/or miRNA-20a on the encapsulated hMSCs, hMSC/hydrogel constructs were evaluated for osteogenic differentiation by measuring ALP activity, which is an important early marker for osteogenic differentiation (FIG. 10c). All groups exhibited a peak ALP activity at 14 days, followed by a decrease in expression levels by day 28. The ALP activity of hMSCs transfected with siNoggin and/or miRNA-20a was significantly higher than that of control group at day 14 and day 21. Since other cells can express the ALP, it is important to examine other specific osteogenic differentiation markers as well. Therefore, quantitative analysis of mRNA expression levels of runt-related transcription factor 2 (Runx2), which is one of the earlier and most specific osteogenic differentiation makers, bone sialoprotein (BSP), which is the later osteogenic differentiation marker, and peroxisome proliferator-activated receptor gamma (PPAR-γ), which is the adipogenic transcription factor, were also evaluated by real-time quantitative reverse transcription-polymerase chain reaction (qRT-PCR) (FIG. 10d,e and f). Compared to the control group, siNoggin and/or miRNA-20a transfected hMSCs expressed significantly higher Runx2 at day 14. Similar to the Runx2 expression, siNoggin and/or miRNA-20a transfected hMSCs expressed significantly higher BSP expression than that in the control group at day 14. However, PPAR-γ expression in the groups treated with siNoggin and/or miRNA-20a was significantly lower than that in the control and cotransfection groups at all time points. These results indicate that siNoggin and/or miRNA-20a transfection could enhance and accelerate the osteogenic differentiation of encapsulated hMSCs.

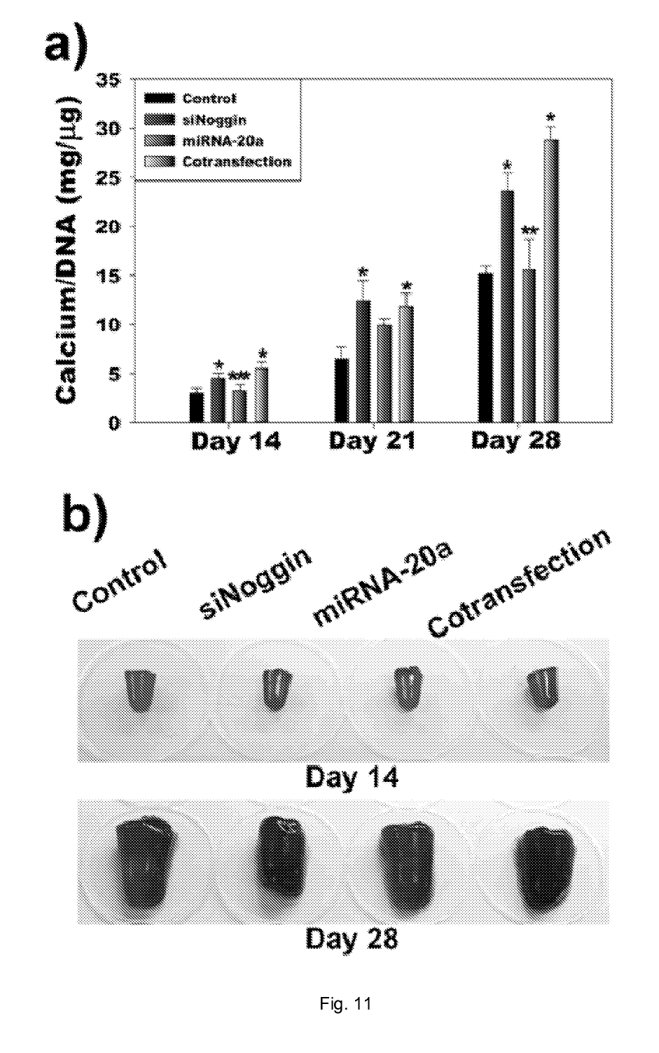

Since the mineralization is the ultimate indicator of osteogenic differentiation of stem cell, the calcium deposition in the hMSC/hydrogel constructs was also evaluated by quantification of calcium content and Alizarin red staining. As shown in FIG. 11, similar to the other osteogenic markers, calcium deposition significantly increased by transfection with siNoggin or cotransfection of siNoggin and miRNA-20a at days 14 and 28, suggesting that only siNoggin accelerates bone-related mineralization of the extracellular environment. Since miRNA-20a had the greater effect on ALP activity, and Runx2 and BSP gene expression compared to control, we hypothesized that the same would be true for calcium deposition. Unlike its effect on other osteogenic differentiation markers, miRNA-20a transfection appeared to have no effect on calcium deposition compared to the control group.

In conclusion, we have engineered in situ forming biodegradable and cytocompatible hydrogels for sustained and localized delivery of siRNA and miRNA for differentiation of encapsulated hMSCs. The biomaterial permitted homogeneous encapsulation of cells and RNA in a mild condition without the need of UV light or a photoinitiator. The swelling and degradation properties of the in situ forming hydrogels were controlled via the density of hydrolysable ester groups in the materials. The biomaterials could preserve bioactivity of RNA during the entire release time. Subsequent delivery of siNoggin or siNoggin/miRNA-20a resulted in enhanced osteogenic differentiation of hMSCs encapsulated within the biomaterials. This system may provide a great platform for controlled and sustained delivery of siRNA or miRNA for regulating stem cell fate for a wide range of applications in tissue regeneration.

All publications and patents mentioned herein are incorporated herein by reference to disclose and describe the specific methods and/or materials in connection with which the publications and patents are cited. The publications and patents discussed herein are provided solely for their disclosure prior to the filing date of the present application. Nothing herein is to be construed as an admission that the present invention is not entitled to antedate such publication or patent by virtue of prior invention. Further, the dates of publication or issuance provided may be different from the actual dates, which may need to be independently confirmed.

Having described the invention, the following is claimed:

1. A composition comprising:
a biodegradable hydrogel that includes a hydrogel forming base polymer and a plurality of physiologically degradable ester linkages, amide linkages, azide-alkyne cycloaddition linkages, acrylate-thiol linkages, urethane linkages, and/or methacrylate-thiol linkages, the hydrogel being cytocompatible and, upon degradation, producing substantially non-toxic products;
a polynucleotide coupling polymeric molecule, the cationic polynucleotide coupling polymeric molecule being coupled to the hydrogel forming base polymer; and
a polynucleotide coupled to the polynucleotide coupling polymeric molecule, the polynucleotide being released under physiological conditions in a spatial and/or temporally controlled or predetermined manner from the composition.

2. The composition of claim 1, the polynucleotide coupling polymeric molecule being covalently linked to the hydrogel forming base polymer.

3. The composition of claim 1, the polynucleotide being electrostatically coupled to the polynucleotide coupling polymeric molecule.

4. The composition claim 1, the polynucleotide being covalently linked to a backbone of the hydrogel.

5. The composition of claim 1, the hydrogel including ester bonds and/or urethane bonds directly linked to photolabile moieties degradable by exposure to ultra-violet radiation.

6. The composition of claim 1, wherein the hydrogel forming base polymer includes a methacrylated or acrylated base polymer.

7. The composition of claim 6, the acrylated or methacrylated hydrogel forming base polymer further including a plurality of physiologically degradable acrylate-thiol or methacrylate-thiol bonds.

8. The composition of claim 1, the hydrogel forming base polymer being selected from the group consisting of dextran (DEX), polyethylene glycol (PEG) and poly(vinyl alcohol) (PVA).

9. The composition of claim 1, the hydrogel forming base polymer being selected from the group consisting of dextran-hydroxyethylmethacrylate (DEX-HEMA), dextran-mono-2-(acryloyloxy)ethyl succinate (DEX-MAES), polyvinyl alcohol-mono-2-(acryloyloxy)ethyl succinate (PVA-MAES), n-arm-polyethylene glycol-mono-2-(acryloyloxy)ethyl succinate (n-arm-PEG-MAES), n-arm-polyethylene glycol-acrylate (n-arm-PEG-A), n-arm-polyethylene glycol-thiol (n-arm-PEG-SH) and n-arm-polyethylene glycol-photolabile moiety-acrylate (n-arm-PEG-PL-A), n-arm-polyethylene glycol-azide (n-arm-PEG-azide), n-arm-polyethylene glycol-alkyne (n-arm-PEG-alkyne) (n=2, 4, 6, 8, 10, etc.).

10. The composition of claim 1, the polynucleotide coupling polymeric molecule being selected from the group consisting of poly(dimethylamino ethyl methacrylate) (pDMAEMA), poly(dimethylamino ethyl methacrylate-cysteamine) (poly(DMAEMA-co-cys)), linear or branched polyethyleneimine (PEI), polyethyleneimine-mono-2-(acryloyloxy)ethyl succinate (PEI-MAES), polyethyleneimine-thiol (PEI-thiol), and polyethyleneimine-glycidyl methacrylate (PEI-GMA), protamine, polylysine, polyamidoamine, polyethyleneimine-photolabile moiety-allyl (PEI-PL-allyl), polyethyleneimine-photolabile moiety-alkyne (PEI-PL-alkyne), polyethyleneimine-photolabile moiety-azide (PEI-PL-azide).

11. The composition of claim 1, the polynucleotide selected from the group consisting of DNA fragments, DNA plasmids, interfering RNA molecules.

12. The composition of claim 1, the polynucleotide comprising siRNA or miRNA.

13. The composition of claim 1, further comprising at least one cell dispersed on or within the biodegradable hydrogel.

14. The composition of claim 13, the at least one cell comprising a progenitor cell.

15. The composition of claim 1, the hydrogel being degraded by hydrolysis of the ester bond linkages.

16. The composition of claim 1, the polynucleotide coupling polymeric molecule being covalently linked to the hydrogel forming polymer by at least one of a covalent hydrolyzable ester, covalent hydrolyzable amide, covalent photodegradable urethane, covalent photodegradable and hydrolyzable ester, or covalent hydrolyzable acrylate-thiol linkage.

17. The composition of claim 1, the hydrogel being photocrosslinked.

18. The composition of claim 1, the hydrogel being formed in situ without photo initiators.

19. The composition of claim 1, the hydrogel being formed via Click chemistry by copper-assisted or copper free azide-alkyne cycloaddition.

20. A composition comprising:
a biodegradable hydrogel that includes a hydrogel forming base polymer and a plurality of physiologically degradable ester linkages, amide linkages, azide-alkyne cycloaddition linkages, acrylate-thiol linkages, urethane linkages, and/or methacrylate-thiol linkages, the hydrogel being cytocompatible and, upon degradation, producing substantially non-toxic products;
a polynucleotide coupling polymeric molecule that is covalently linked to the hydrogel forming base polymer; and
a polynucleotide coupled to the polynucleotide coupling polymeric molecule, the polynucleotide being released under physiological conditions in a spatial and/or temporally controlled or predetermined manner from the composition.

21. The composition of claim 20, the polynucleotide being electrostatically coupled to the polynucleotide coupling polymeric molecule.

22. The composition claim 20, the polynucleotide being covalently linked to a backbone of the hydrogel.

23. The composition of claim 20, the hydrogel including ester bonds and/or urethane bonds directly linked to photolabile moieties degradable by exposure to ultra-violet radiation.

24. The composition of claim 20, wherein the hydrogel forming base polymer includes a methacrylated or acrylated base polymer.

25. The composition of claim 24, the acrylated or methacrylated hydrogel forming base polymer further including a plurality of physiologically degradable acrylate-thiol or methacrylate-thiol bonds.

26. The composition of claim 20, the hydrogel forming base polymer being selected from the group consisting of dextran (DEX), polyethylene glycol (PEG) and poly(vinyl alcohol) (PVA).

27. The composition of claim 20, the hydrogel forming base polymer being selected from the group consisting of dextran-hydroxyethylmethacrylate (DEX-HEMA), dextran-mono-2-(acryloyloxy)ethyl succinate (DEX-MAES), polyvinyl alcohol-mono-2-(acryloyloxy)ethyl succinate (PVA-MAES), n-arm-polyethylene glycol-mono-2-(acryloyloxy)ethyl succinate (n-arm-PEG-MAES), n-arm-polyethylene glycol-acrylate (n-arm-PEG-A), n-arm-polyethylene glycol-thiol (n-arm-PEG-SH) and n-arm-polyethylene glycol-photolabile moiety-acrylate (PEG-PL-A), n-arm-polyethylene glycol-azide (n-arm-PEG-azide), n-arm-polyethylene glycol-alkyne (n-arm-PEG-alkyne) (n=2, 4, 6, 8, 10, etc.).

28. The composition of claim 20, the polynucleotide coupling polymeric molecule selected from the group consisting of poly(dimethylamino ethyl methacrylate-cysteamine) (poly(DMAEMA-co-cys)), polyethyleneimine-mono-2-(acryloyloxy)ethyl succinate (PEI-MAES), polyethyleneimine-thiol (PEI-thiol), and polyethyleneimine-glycidyl methacrylate (PEI-GMA), polyethyleneimine-photolabile moiety-allyl (PEI-PL-allyl), polyethyleneimine-photolabile moiety-alkyne (PEI-PL-alkyne), polyethyleneimine-photolabile moiety-azide (PEI-PL-azide), allylated/acrylated/methacrylated/thiolated modified poly(dimethylamino ethyl methacrylate) (pDMAEMA), linear or branched polyethyleneimine (PEI), protamine, polylysine, polyamidoamine.

29. The composition of claim 20, the polynucleotide selected from the group consisting of DNA fragments, DNA plasmids, interfering RNA molecules.

30. The composition of claim 20, the polynucleotide comprising siRNA or miRNA.

31. The composition of claim 20, further comprising at least one cell dispersed on or within the photocrosslinked or in situ gelling biodegradable hydrogel.

32. The composition of claim 31, the at least one cell comprising a progenitor cell.

33. The composition of claim 20, the hydrogel being degraded by hydrolysis of the ester bond linkages.

34. The composition of claim 20, the polynucleotide coupling polymeric molecule being covalently linked to the hydrogel forming polymer by at least one of a covalent hydrolyzable ester, covalent hydrolyzable amide, covalent photodegradable urethane, covalent photodegradable and hydrolyzable ester, or covalent hydrolyzable acrylate-thiol linkage.

35. The composition of claim 20, the hydrogel being photocrosslinked.

36. The composition of claim 20, the hydrogel being formed in situ without photo initiators.

37. The composition of claim 20, the hydrogel being formed via Click chemistry by copper-assisted or copper free azide-alkyne cycloaddition.

38. A composition comprising:
an in situ formed biodegradable hydrogel that includes a hydrogel forming base polymer and a plurality of physiologically degradable ester linkages, amide linkages, azide-alkyne cycloaddition linkages, acrylate-thiol linkages, urethane linkages, and/or methacrylate-thiol linkages, the hydrogel being cytocompatible and, upon degradation, producing substantially non-toxic products;
a polynucleotide coupling polymeric molecule, the cationic polynucleotide coupling polymeric molecule being coupled to the hydrogel forming base polymer; and
a polynucleotide coupled to the polynucleotide coupling polymeric molecule, the polynucleotide being released under physiological conditions in a spatial and/or temporally controlled or predetermined manner from the composition.

39. The composition of claim 38, the polynucleotide coupling polymeric molecule being covalently linked to the hydrogel forming base polymer.

40. The composition of claim 38, the polynucleotide being electrostatically coupled to the polynucleotide coupling polymeric molecule.

41. The composition claim 38, the polynucleotide being covalently linked to the a backbone of the hydrogel.

42. The composition of claim 38, the hydrogel further including ester bonds and/or urethane bonds directly linked to photolabile moieties degradable by exposure to ultra-violet radiation.

43. The composition of claim 38, wherein the hydrogel forming base polymer includes a methacrylated or acrylated base polymer.

44. The composition of claim 43, the acrylated or methacrylated hydrogel forming base polymer further including a plurality of physiologically degradable acrylate-thiol or methacrylate-thiol bonds.

45. The composition of claim 38, the hydrogel forming base polymer being selected from the group consisting of dextran (DEX), polyethylene glycol (PEG) and poly(vinyl alcohol) (PVA).

46. The composition of claim 38, the hydrogel forming base polymer being selected from the group consisting of dextran-hydroxyethylmethacrylate (DEX-HEMA), dextran-mono-2-(acryloyloxy)ethyl succinate (DEX-MAES), polyvinyl alcohol-mono-2-(acryloyloxy)ethyl succinate (PVA-MAES), n-arm-polyethylene glycol-mono-2-(acryloyloxy)ethyl succinate (n-arm-PEG-MAES), n-arm-polyethylene glycol-acrylate (n-arm-PEG-A), n-arm-polyethylene glycol-thiol (n-arm-PEG-SH) and n-arm-polyethylene glycol-photolabile moiety-acrylate (PEG-PL-A), n-arm-polyethylene glycol-azide (n-arm-PEG-azide), n-arm-polyethylene glycol-alkyne (n-arm-PEG-alkyne) (n=2, 4, 6, 8, 10, etc.).

47. The composition of claim 38, the nucleic acid coupling polymeric molecule being selected from the group consisting of poly(dimethylamino ethyl methacrylate) (pDMAEMA), poly(dimethylamino ethyl methacrylate-cysteamine) (poly(DMAEMA-co-cys)), linear or branched polyethyleneimine (PEI), polyethyleneimine-mono-2-(acryloyloxy)ethyl succinate (PEI-MAES), polyethyleneimine-thiol (PEI-thiol), and polyethyleneimine-glycidyl methacrylate (PEI-GMA), protamine, polylysine, polyamidoamine, polyethyleneimine-photolabile moiety-allyl (PEI-PL-allyl), polyethyleneimine-photolabile moiety-alkyne (PEI-PL-alkyne), polyethyleneimine-photolabile moiety-azide (PEI-PL-azide).

48. The composition of claim 38, the polynucleotide selected from the group consisting of DNA fragments, DNA plasmids, interfering RNA molecules.

49. The composition of claim 38, the polynucleotide comprising siRNA or miRNA.

50. The composition of claim 38, further comprising at least one cell dispersed on or within the in situ formed biodegradable hydrogel.

51. The composition of claim 38, the at least one cell comprising a progenitor cell.

52. The composition of claim 38, the hydrogel being degraded by hydrolysis of the ester bond linkages.

53. The composition of claim 38, the polynucleotide coupling polymeric molecule being covalently linked to the hydrogel forming polymer by at least one of a covalent hydrolyzable ester, covalent hydrolyzable amide, covalent photodegradable urethane, covalent photodegradable and hydrolyzable ester, or covalent hydrolyzable acrylate-thiol linkage.

54. A composition comprising:
a photocrosslinked biodegradable hydrogel that includes a hydrogel forming base polymer and a plurality of physiologically degradable ester linkages, amide linkages, azide-alkyne cycloaddition linkages, acrylate-thiol linkages, urethane linkages, and/or methacrylate-thiol linkages, the hydrogel being cytocompatible and, upon degradation, producing substantially non-toxic products;
a polynucleotide coupling polymeric molecule, the cationic polynucleotide coupling polymeric molecule being coupled to the hydrogel forming base polymer; and
a polynucleotide coupled to the polynucleotide coupling polymeric molecule, the polynucleotide being released under physiological conditions in a spatial and/or temporally controlled or predetermined manner from the composition.

55. The composition of claim 54, the polynucleotide coupling polymeric molecule being covalently linked to the hydrogel forming base polymer.

56. The composition of claim 54, the polynucleotide being electrostatically coupled to the polynucleotide coupling polymeric molecule.

57. The composition claim 54, the polynucleotide being covalently linked to a backbone of the hydrogel.

58. The composition of claim 54, the hydrogel including ester bonds and/or urethane bonds directly linked to photolabile moieties degradable by exposure to ultra-violet radiation.

59. The composition of claim 54, wherein the hydrogel forming base polymer includes a methacrylated or acrylated base polymer.

60. The composition of claim 59, the acrylated or methacrylated hydrogel forming base polymer further including a plurality of physiologically degradable acrylate-thiol or methacrylate-thiol bonds.

61. The composition of claim 54, the hydrogel forming base polymer being selected from the group consisting of dextran (DEX), polyethylene glycol (PEG) and poly(vinyl alcohol) (PVA).

62. The composition of claim 54, the hydrogel forming base polymer being selected from the group consisting of dextran-hydroxyethylmethacrylate (DEX-HEMA), dextran-mono-2-(acryloyloxy)ethyl succinate (DEX-MAES), polyvinyl alcohol-mono-2-(acryloyloxy)ethyl succinate (PVA-MAES), n-arm-polyethylene glycol-mono-2-(acryloyloxy)ethyl succinate (n-arm-PEG-MAES), n-arm-polyethylene glycol-acrylate (n-arm-PEG-A), n-arm-polyethylene glycol-thiol (n-arm-PEG-SH) and n-arm-polyethylene glycol-photolabile moiety-acrylate (PEG-PL-A), n-arm-polyethylene glycol-azide (n-arm-PEG-azide), n-arm-polyethylene glycol-alkyne (n-arm-PEG-alkyne) (n=2, 4, 6, 8, 10, etc.).

63. The composition of claim 54, the nucleic acid coupling polymeric molecule being selected from the group consisting of poly(dimethylamino ethyl methacrylate) (pDMAEMA), poly(dimethylamino ethyl methacrylate-cysteamine) (poly(DMAEMA-co-cys)), linear or branched polyethyleneimine (PEI), polyethyleneimine-mono-2-(acryloyloxy)ethyl succinate (PEI-MAES), polyethyleneimine-thiol (PEI-thiol), and polyethyleneimine-glycidyl methacrylate (PEI-GMA), protamine, polylysine, polyamidoamine, polyethyleneimine-photolabile moiety-allyl (PEI-PL-allyl), polyethyleneimine-photolabile moiety-alkyne (PEI-PL-alkyne), polyethyleneimine-photolabile moiety-azide (PEI-PL-azide).

64. The composition of claim 54, the polynucleotide selected from the group consisting of DNA fragments, DNA plasmids, interfering RNA molecules.

65. The composition of claim 54, the polynucleotide comprising siRNA or miRNA.

66. The composition of claim 54, further comprising at least one cell dispersed on or within the photocrosslinked biodegradable hydrogel.

67. The composition of claim 66, the at least one cell comprising a progenitor cell.

68. The composition of claim 54, the hydrogel being degraded by hydrolysis of the ester bond linkages.

69. The composition of claim 54, the polynucleotide coupling polymeric molecule being covalently linked to the hydrogel forming polymer by at least one of a covalent hydrolyzable ester, covalent hydrolyzable amide, covalent photodegradable urethane, covalent photodegradable and hydrolyzable ester, or covalent hydrolyzable acrylate-thiol linkage.

70. A composition comprising:
a biodegradable hydrogel that includes a hydrogel forming base polymer and a plurality of physiologically degradable ester linkages, amide linkages, azide-alkyne cycloaddition linkages, acrylate-thiol linkages, urethane linkages, and/or methacrylate-thiol linkages, the hydrogel being cytocompatible and, upon degradation, producing substantially non-toxic products; and
a polynucleotide covalently linked to the hydrogel forming base polymer, the polynucleotide being released under physiological conditions in a spatial and/or temporally controlled or predetermined manner from the composition.

71. The composition of claim 70, the hydrogel further including ester bonds and/or urethane bonds being directly linked to photolabile moieties degradable by exposure to ultra-violet radiation.

72. The composition of claim 70, wherein the hydrogel forming base polymer includes a methacrylated or acrylated base polymer.

73. The composition of claim 72, the acrylated or methacrylated hydrogel forming base polymer further including a plurality of physiologically degradable acrylate-thiol or methacrylate-thiol bonds.

74. The composition of claim 70, the hydrogel forming base polymer being selected from the group consisting of dextran (DEX), polyethylene glycol (PEG) and poly(vinyl alcohol) (PVA).

75. The composition of claim 70, the hydrogel forming base polymer being selected from the group consisting of dextran-hydroxyethylmethacrylate (DEX-HEMA), dextran-mono-2-(acryloyloxy)ethyl succinate (DEX-MAES), polyvinyl alcohol-mono-2-(acryloyloxy)ethyl succinate (PVA-MAES), n-arm-polyethylene glycol-mono-2-(acryloyloxy)ethyl succinate (n-arm-PEG-MAES), n-arm-polyethylene glycol-acrylate (n-arm-PEG-A), n-arm-polyethylene glycol-thiol (n-arm-PEG-SH) and n-arm-polyethylene glycol-photolabile moiety-acrylate (PEG-PL-A), n-arm-polyethylene glycol-azide (n-arm-PEG-azide), n-arm-polyethylene glycol-alkyne (n-arm-PEG-alkyne) (n=2, 4, 6, 8, 10, etc.).

76. The composition of claim 70, the polynucleotide selected from the group consisting of DNA fragments, DNA plasmids, interfering RNA molecules.

77. The composition of claim 70, the polynucleotide comprising siRNA or miRNA.

78. The composition of claim 70, further comprising at least one cell dispersed on or within the photocrosslinked or in situ gelling biodegradable hydrogel.

79. The composition of claim 78, the at least one cell comprising a progenitor cell.

80. The composition of claim 70, the hydrogel being degraded by hydrolysis of the ester bond linkages.

81. The composition of claim 70, the polynucleotide coupling polymeric molecule being covalently linked to the hydrogel forming polymer by at least one of a covalent hydrolyzable ester, covalent hydrolyzable amide, covalent photodegradable urethane, covalent photodegradable and hydrolyzable ester, or covalent hydrolyzable acrylate-thiol linkage.

82. The composition of claim 70, the hydrogel being photocrosslinked.

83. The composition of claim 70, the hydrogel being formed in situ without photo initiators.

84. The composition of claim 70, the hydrogel being formed via Click chemistry by copper-assisted or copper free azide-alkyne cycloaddition.

85. A composition comprising:
a hydrogel that includes a hydrogel forming base polymer and a plurality of physiologically degradable ester linkages, amide linkages, azide-alkyne cycloaddition linkages, acrylate-thiol linkages, urethane linkages, and/or methacrylate-thiol linkages, the hydrogel being cytocompatible and, upon degradation, producing substantially non-toxic products; and
a polynucleotide provided in or coupled to the hydrogel, the polynucleotide being released under physiological conditions in a spatial and/or temporally controlled or predetermined manner from the composition.

86. The composition of claim 85, the hydrogel further including ester bonds and/or urethane bonds being directly linked to photolabile moieties degradable by exposure to ultra-violet radiation.

87. The composition of claim 85, wherein the hydrogel forming base polymer includes a methacrylated or acrylated base polymer.

88. The composition of claim 87, the acrylated or methacrylated hydrogel forming base polymer further including a plurality of physiologically degradable acrylate-thiol or methacrylate-thiol bonds.

89. The composition of claim 85, the hydrogel forming base polymer being selected from the group consisting of dextran (DEX), polyethylene glycol (PEG) and poly(vinyl alcohol) (PVA).

90. The composition of claim 85, the hydrogel forming base polymer being selected from the group consisting of dextran-hydroxyethylmethacrylate (DEX-HEMA), dextran-mono-2-(acryloyloxy)ethyl succinate (DEX-MAES), polyvinyl alcohol-mono-2-(acryloyloxy)ethyl succinate (PVA-MAES), n-arm-polyethylene glycol-mono-2-(acryloyloxy)ethyl succinate (n-arm-PEG-MAES), n-arm-polyethylene glycol-acrylate (n-arm-PEG-A), n-arm-polyethylene glycol-thiol (n-arm-PEG-SH) and n-arm-polyethylene glycol-photolabile moiety-acrylate (PEG-PL-A), n-arm-polyethylene glycol-azide (n-arm-PEG-azide), n-arm-polyethylene glycol-alkyne (n-arm-PEG-alkyne) (n=2, 4, 6, 8, 10, etc.).

91. The composition of claim 85, the polynucleotide selected from the group consisting of DNA fragments, DNA plasmids, interfering RNA molecules.

92. The composition of claim 85, the polynucleotide comprising siRNA or miRNA.

93. The composition of claim 85, further comprising at least one cell dispersed on or within the hydrogel.

94. The composition of claim 92, the at least one cell comprising a progenitor cell.

95. The composition of claim 85, the hydrogel being degraded by hydrolysis of the ester bond linkages.

96. The composition of claim 85, the polynucleotide coupling polymeric molecule being covalently linked to the hydrogel forming polymer by at least one of a covalent hydrolyzable ester, covalent hydrolyzable amide, covalent photodegradable urethane, covalent photodegradable and hydrolyzable ester, or covalent hydrolyzable acrylate-thiol linkage.

97. The composition of claim 85, the hydrogel being photocrosslinked.

98. The composition of claim 85, the hydrogel being formed in situ without photo initiators.

99. The composition of claim 85, the hydrogel being formed via Click chemistry.
